SEQUENCE LISTING (17 sequences)

SEQ ID NO: 1 - DNA, 2164 bp, Petroselinum crispum
atggaaaatggtaatggtgctacaacaaatggtcatgttaatggtaatggaatggatttttgtatgaaaacagaagatccattatattggggtattgctgctgaagctatgacaggttcacatttagatgaagttaaaaaaatggttgctgaatatcgtaaaccagttgttaaattaggtggtgaaacattaacaatttcacaagttgctgctatttcagctcgtgatggttcaggtgttacagttgaattatcagaagctgctagagctggagttaaagcatcatcagattgggttatggattcaatgaataaaggtacagattcatatggtgttacaacaggttttggtgctacatcacatcgtcgtacaaaacaaggtggtgctttacaaaaagaacttattcgttttttaaatgctggtatttttggtaatggttcagataatacattaccacattcagctacacgtgctgctatgttagttcgtattaatacattattacaaggttattcaggtattcgttttgaaattttagaagctattacaaaatttcttaatcaaaatattacaccatgtttaccattacgtggtacaattacagcttcaggtgatttagttccattatcatatattgctggtttattaacaggtcgtccaaattcaaaagctgttggtccaactggtgttattttatcaccagaagaagcatttaaattagctggtgttgaaggtggtttttttgaattacaaccaaaagaaggtttagctttagttaatggtacagctgttggttcaggtatggcttcaatggttttatttgaagctaatattcttgctgttttagcagaagttatgtctgctatttttgctgaagttatgcaaggtaaaccagaatttacagatcatttaacacacaaattaaaacatcatccaggtcaaattgaagctgctgctattatggaacacattcttgatggttcagcttatgttaaagctgctcaaaaattacatgaaatggacccattacaaaaaccaaaacaagatcgttatgctttacgtacatcaccacaatggttaggtccacaaattgaagttattcgttcatcaacaaaaatgattgaacgtgaaattaattcagttaatgataatccattaattgatgtttcacgtaataaagctattcatggtggtaattttcaaggtacaccaattggagtttcaatggataatacacgtttagctattgctgctattggtaaattaatgtttgctcaattttcagaattagttaatgatttttataataatggtttaccatcaaatttatcaggtggtcgtaatccatcattagattatggttttaaaggtgctgaaattgctatggcttcatattgttcagaattacaatttttagctaatccagttacaaatcatgttcaatcagctgaacaacataatcaagatgttaattcattaggtttaatttcatcacgtaaaacatcagaagctgttgaaattttaaaattaatgtcaacaacatttttagttggtttatgtcaagctattgatcttcgtcatttagaagaaaatttaaaatcaacagttaaaaatacagtttcatcagttgctaaacgtgttttaacaatgggtgttaatggtgaattacatccatcacgtttttgtgaaaaagaccttcttcgtgttgttgatcgtgaatatatttttgcttatattgatgatccatgttcagctacatatccattaatgcaaaaattacgtcaaacattagttgaacatgctttaaaaaatggtgataatgaacgtaatttatcaacatcaatttttcaaaaaattgctacatttgaagatgaattaaaagcattattaccaaaagaagttgaatcagctagagctgctttagaatcaggtaatccagctattccaaatcgtattgaagaatgtcgttcatatccattatataaatttgttcgtaaagaattaggtacagaatatttaacaggtgaaaaagttacatcaccaggtgaagaatttgaaaaagtttttattgctatgtcaaaaggtgaaattattgatccattattagaatgtttagaatcatggaatggtgctccattaccaatttgttaatagaagcttctcg

SEQ ID NO: 2 - protein, 716 aa, Petroselinum crispum
MENGNGATTNGHVNGNGMDFCMKTEDPLYWGIAAEAMTGSHLDEVKKMVAEYRKPVVKLGGETLTISQVAAISARDGSGVTVELSEAARAGVKASSDWVMDSMNKGTDSYGVTTGFGATSHRRTKQGGALQKELIRFLNAGIFGNGSDNTLPHSATRAAMLVRINTLLQGYSGIRFEILEAITKFLNQNITPCLPLRGTITASGDLVPLSYIAGLLTGRPNSKAVGPTGVILSPEEAFKLAGVEGGFFELQPKEGLALVNGTAVGSGMASMVLFEANILAVLAEVMSAIFAEVMQGKPEFTDHLTHKLKHHPGQIEAAAIMEHILDGSAYVKAAQKLHEMDPLQKPKQDRYALRTSPQWLGPQIEVIRSSTKMIEREINSVNDNPLIDVSRNKAIHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTSEAVEILKLMSTTFLVGLCQAIDLRHLEENLKSTVKNTVSSVAKRVLTMGVNGELHPSRFCEKDLLRVVDREYIFAYIDDPCSATYPLMQKLRQTLVEHALKNGDNERNLSTSIFQKIATFEDELKALLPKEVESARAALESGNPAIPNRIEECRSYPLYKFVRKELGTEYLTGEKVTSPGEEFEKVFIAMSKGEIIDPLLECLESWNGAPLPIC

SEQ ID NO: 3 - protein, 715 aa, Daucus carota
MAYTNGHHENGNGVDLCMKKEDPLSWGVAAEALTGSHLEEVKRMVAEYRKPVVKLGGETLTISQVAAISARDDSGVKVELSEAARAGVKASSDWVMESMNKGTDSYGVTTGFGATSHRRTKQGGALQKELIRFLNAGIFGSGAEAGNNTLPHSATRAAMLVRINTLLQGYSGIRFEILEAITKFLNHNITPCLPLRGTITASGDLVPLSYIAGLLTGRPNSKAVGPTGVTLSPEEAFKLAGVEGGFFELQPKEGLALVNGTAVGSGMASMVLFEANILAVLAEVMSAIFAEVMQGKPEFTDHLTHKLKHHPGQIEAAAIMEHILDGSAYVKAAQKLHEMDPLQKPKQDRYALRTSPQWLGPQIEVIRSSTKMIEREINSVNDNPLIDVSRNKAIHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTSEAVEILKLMSTTFLVGLCQAIDLRHLEENMKSTVKNTVSQVAKRVLTMGVNGELHPSRFCEKDLLRVVDREYIFAYIDDPCSATYPLMQKLRETLVEHALNNGDKERNLSTSIFQKIAAFEDELKALLPKEVESARAAVESGNPAIPNRIKECRSYPLYKFVREELGTEYLTGEKVTSPGEEFDKVFTAMTKGEIIDPLLECLQSWNGAPLPIC

SEQ ID NO: 4 - protein, 716 aa, Catharanthus roseus
MKNMGSTENGHINNGEVLDFCLKDPLNWGMAADSLKGSHLDEVKRMVAEFRKPVVKLGGETLTISQVAAIAARDYNAVKVELSEDARAGVKASSDWVMESMNKGTDSYGVTTGFGATSHRRTKQGGALQKELIRFLNAGIFGNGTESSHTLPHSATRAAMLVRINTLLQGYSGIRFEILEAITKFLNHNITPCLPLRGTITASGDLVPLSYIAGLLTGRPNSKAVGPNGEIVNPEQAFKMAGVNDGLFELQPKEGLALVNGTAVGSGMASMVLFEANILAVLSEVLSAIFAEVMNGKPEFTDHLTHKLKHHPGQIEAAAIMEHILDGSGYVKAAHKLHEMDPLQKPKQDRYALRTSPQWLGPQIEVIRSATKMIEREINSVNDNPLIDVSRNKALHGGNFQGTPIGVSMDNARLAIASIGKLMFAQISELVNDFYNNGLPSNLSGGRNPSLVYGFKGAEIAMASYCSELQYLANPVTNHVQSAEQHNQDVNSLGLISSRKTAEAVEILKLMSSTYLVALCQAIDLRHLEENLKNAVKNTVSQVAKRTLTVGVNGELHPSRFCEKDLIRVVDREYVFAYVDDPCSGTYPLMEKLRQVIVDHALQNGESEKNVNTSIFQKIAAFEDELKTVLPKEVESARTALENGNPAIPNRIKECRSYPLYKFVREDVGAEFLTGEKDRSPGEEFDKVFTAMCNEKIIDPLLECLKEWNGAPLPIC

SEQ ID NO: 5 - protein, 720 aa, Rudbeckia hirta
MENGNHVNGNHVNGNGAAAEFCIKDPLNWGAAAEALTGSHLDEVKKMVAEFRKPVVKLGGETLTVSQVAGIAAAGGGGGGTVTVELSEAARAGVKASSDWVMESMNKGTDSYGVTTGFGATSHRRTKNGGALQKELIRFLNAGIFGNGTESSHTLPHSATRAAMLVRINTLLQGYSGIRFEILEAITKFLNNNITPCLPLRGTITASGDLVPLSYIAGLLTGRPNSKAMGPTGEILNAETAFAQAGVKGGFFELQPKEGLALVNGTAVGSGMASMVLFEANVLALLSEVLSAIFAEVMQGKPEFTDHLTHKLKHHPGQIEAAAIMEYILDGSDYVKAAQKVHEMDPLQKPKQDRYALRTSPQWLGPQIEVIRSATKMIEREINSVNDNPLIDVSRNKALHGGNFQGTPIGVSMDNTRLAIAAIGKLMFAQFSELVNDFYNNGLPSNLSGGRNPSLDYGFKGGEIAMASYCSELQFLANPVTNHVQSAEQHNQDVNSLGLISARKTAEAVEILKLMSSTYLVALCQSVDLRHLEENMKSTVKNTVSQVAKKVLTMGVNGELHPSRFCEKDLLRVVDREYVFAYIDDPCLATYPLMQKLRQVLVDHALNNGETEKNTNTSIFQKIATFEDELKAILPKEVESVRVAFENGTMSIPNRIKACRSYPLYRFVREELSGAYLTGEKVTSPGEEFDKVFTAMSKGQIIDPLLECVEGWNGAPLPIC

SEQ ID NO: 6 - protein, 711 aa, Lactuca sativa
MENGNHVNGVVNELCIKDPLNWGVAAEALTGSHLDEVKKMVAEFRKPVVKLGGETLTVSQVAGIAAANDSDTVKVELSEAARAGVKASSDWVMESMNKGTDSYGVTTGFGATSHRRTKQGGALQKELIRFLNAGIFGNGTETSHTLPHSATRAAMIVRINTLLQGYSGIRFEILEAITKFLNNNITPCLPLRGTITASGDLVPLSYIAGLLTGRPNSKAVGPTGEVLNAEKAFAAAGVEGGFFELQPKEGLALVNGTAVGSGMASMVLFDANVLALLSEVLSAIFAEVMQGKPEFTDHLTHKLKHHPGQIEAAAIMEYILDGSDYVKAAQKVHEMDPLQKPKQDRYALRTSPQWLGPQIEVIRSSTKMIEREINSVNDNPLIDVSRNKALHGGNFQGTPIGVSMDNTRLAIAAIGKLMFAQFSELVNDFYNNGLPSNLSGGRNPSLDYGFKGGEIAMASYCSELQFLANPVTNHVQSAEQHNQDVNSLGLISARKTAEAVDILKLMSSTYLVALCQSIDLRHLEENMKSTVKNTVSQVAKKVLTMGVNGELHPSRFCEKDLLRVVDREYVFAYIDDVCSGTYPLMQKLRQVLVDHALNNGETEKNTNTSIFQKIATFEEELKVLLPKEVEGVRIAYENDTLSIPNRIKACRSYPLYRFVREELGRGFLTGEKVTSPGEEFDRVFTAMCKGQIIDPLLECLGGWNGEPLPIC

SEQ ID NO: 7 - protein, 161 aa, Listeria monocytogenes
FAEGKDNVTPFEFIPWILGQCATVKEARRLLQRINLVNISFSENLPLSPLHWLMADQTESIVVECVKDGLHIYDNPVGVLTNNPTFDYQLFNLNNYRVLSSETPENNFSKEIDLDAYSRGMGGIGLPGDLSSMSRFVKATFTKLNSVSGDSESESISQFFH

SEQ ID NO: 8 - DNA, 486 bp, Listeria monocytogenes
atatggaaaaattggctaatactttctgattcagaatcacctgaaacagaattcaatttggtaaaagttgctttcacaaaacgagacatagaagataaatcaccaggtaagccaattccgcccatcccacgactataagcatccaaatcaatctctttggaaaaattattttctggggtttcactcgaaagaacgcgataattgtttaaattaaatagttggtaatcaaatgttggattatttgttaacacgccaacaggattatcataaatgtgaagtccatctttcacacattccactacaatagattctgtttgatcagccatcaaccaatgtaatggagacagcggtaaattttcactaaaactaatattcacgagattgattctctgaagtaatcttcttgcttcttttacagtagcgcattgaccaagaatccacggaataaattcaaagggggtcacattgtccttaccttctgcaaaa

SEQ ID NO: 9 - protein, 465 aa, Lactobacillus brevis
MQSSLKKSLYLGLAALSFAGVAAVSTTASAKSYATAGAYSTLKTDAATRNVEATGTNALYTKPGTVKGAKVVASKATMAKLASSKKSADYFRAYGVKTTNRGSVYYRVVTMDGKYRGYVYGGKSDTAFAGGIKSAETTTKADMPARTTGFYLTDTSKNTLWTAPKYTQYKASKVSLYGVAKDTKFTVDQAATKTREGSLYYHVTATNGSGISGWIYAGKGFSTTATGTQVLGGLSTDKSVTATNDNSVKIVYRTTDGTQVGSNTWVTSTDGTKAGSKVSDKAADQTALEAYINANKPSGYTVTNPNAADATYGNTVYATVSQAATSKVALKVSGTPVTTALTTADANDKVAANDTTANGSSVAGSTVYAAGTKLAQLTTDLTGEKGQVVTLTAIDTDLEDATFTGTTTYYSDLGKAYHYTYTYNKDSAASSNASTQFGSNVTGTLTATLVMGKSTATANGTTWFN

SEQ ID NO: 10 - DNA, 1763 bp, Lactobacillus brevis
gattacaaaggctttaagcaggttagtgacgttttagttatgtaacaataacattacaggacacccataattgtttcaatccaacgacaatcagagcgtaatccttgtatctccttaaggaaatcgctatacttatcttcgtagttaggggatagctgatcgggtccgctaatgttatgaaataaaattcttaacaaaagcgctaacttcggttatactattcttgcttgataaattacatattttatgtttggaggaagaaagattatgcaatcaagtttaaagaaatctctttacttgggccttgccgcattgagctttgctggtgttgctgccgtttcaacgactgcttcagctaagtcatacgctactgcaggtgcctattcaacgttaaagacggacgctgctactcgtaacgtcgaagctactggtactaacgctttatacacgaagccaggtactgttaagggtgctaaggttgtcgcttctaaggctactatggctaagttagcttcttcaaagaagtcagctgactacttccgtgcttacggtgttaagaccactaaccgtggttcagtttactaccgtgttgtaacgatggatggcaagtaccgtggttacgtttatggtggcaagtctgacactgcctttgctggtggtatcaagtctgctgaaacgactactaaggctgatatgcctgcacgtactactgggttctacttaactgacacttcaaagaacactctttggacggctcctaagtacactcaatacaaggcaagtaaagttagcctttatggtgttgctaaggacaccaagtttactgtagatcaggctgctactaagactcgtgaaggttcattatactatcacgtaactgctactaacggtagtggtattagtggttggatttacgctggtaagggcttcagtactactgctactggtacacaagtacttggtggtctgtcaactgataagtcagttacagcaaccaacgataacagtgttaagattgtttaccgtacgactgatggcactcaagttggttctaacacttgggtaacttcaactgatggtacaaaggcaggttctaaggtaagcgataaggccgccgatcaaactgctcttgaagcctacatcaatgctaacaagcctagcggttacactgtaactaaccctaatgctgcagatgctacctatggtaacacagtttacgctactgtttcccaagcagctacttctaaggtcgctttgaaggtctcagggactcctgttactactgcattgactacagctgatgctaatgataaggttgcagctaacgataccactgctaatggtagttctgttgcaggctcaacagtctatgctgctggtactaagttggctcaattaacaactgacttgactggtgaaaagggtcaagttgtcacattaactgccatcgatactgatttggaagacgctacgttcactggaactacgacttactattcagatcttggtaaagcataccactacacttacacttacaataaggacagtgctgcttcttcaaatgcaagtacccaatttggttcaaacgtcactggtactttaactgctacccttgttatgggtaagtctactgctactgctaacggtactacttggttcaactaataattattatttaggtgagctttgttgataaaaaggtcttttcaacgtttatgttggggagacctttttatattgaaaaaattaggcctttgttagga

SEQ ID NO: 11 - protein, 461 aa, Lactococcus lactis
MKKKIISAILMSTVILSAAAPLSGVYADTNSDIAKQDATISSAQSAKAQAQAQVDSLQSKVDSLQQKQTSTKAQIAKIESEAKALNAQIATLNESIKERTKTLEAQARSAQVNSSATNYMDAVVNSKSLTDVIQKVTAIATVSSANKQMLEQQEKEQKELSQKSETVKKNYNQFVSLSQSLDSQAQELTSQQAELKVATLNYQATIATAQDKKQALLDEKAAAEKAAQEAAKKQAAYEAQQKEAAQAQAASTAATAKAVEAATSSASASSSQAPQVSTSTDNTTSNASASNSSNSSSNSSSSSSSSSSSSSSSSNSNAGGNTNSGTSTGNTGGTTTGGSGINSSPIGNPYAGGGCTDYVWQYFAAQGIYIRNIMPGNGGQWASNGPAQGVLHVVGAAPGVIASSFSADFVGYANSPYGHVAIVKSVNSDGTITIKEGGYGTTWWGHERTVSASGVTFLMPN

SEQ ID NO: 12 - DNA, 2016 bp, Lactococcus lactis
ggtaccagtgagtcatttatttttgatttttctaatgtatagtgtcactcagcacaggccaagctatatcattatcttagcggcgattttgggtgcaatctatgatagctattatttaggtatttacggaattgcgaccttattgttcccacttattgctctttttgtatataatatacaaataactatatttactaatcgctggacaaggctttttacaacaattattattgtgaccgcttttgaagtttttagtgcaatcattatgacagcttttggatttgcccaacttcagtttatcaaatttgttgtttaccagttagcgcctacacttttgctcaatattatcttagctgtagccttacaattccctttagaaatcttttacagattaaagaaaagtcatgtaagatacaattagaaagtgttttgtaatcataaagaaatattaaggtggggtaggaatagtataatatgtttattcaaccgaacttaatgggaggaaaaattaaaaaagaacagttatgaaaaaaaagattatctcagctattttaatgtctacagtgatactttctgctgcagccccgttgtcaggtgtttacgctgacacaaactcagatattgctaaacaagatgcgacaatttcaagcgcgcaatctgctaaagcacaagcacaagcacaagttgatagcttgcaatcaaaagttgacagcttacaacaaaagcaaacaagtactaaagcacaaatcgctaaaatcgaaagcgaagctaaagcacttaatgctcaaattgctactttgaacgaaagtatcaaagaacgtacaaagacattggaagctcaagcacgtagtgctcaagttaacagctcagcaacaaattatatggatgctgttgttaattcaaaatctttgacagatgttattcaaaaagtaacagctattgctactgtttctagtgccaacaaacaaatgttggaacaacaagaaaaagagcaaaaagagcttagccaaaagtcagaaactgttaaaaagaactacaaccagttcgtttctctttcacaaagtttggattctcaagctcaagaattgacttcacaacaagctgaactcaaagttgcgactttgaactatcaagcaacaattgcaactgcgcaagataaaaaacaagctttattagatgaaaaagcagctgcagaaaaagcagctcaagaagcagctaaaaaacaagcggcttatgaagctcaacaaaaagaagcagcacaagcacaagcagcttcaacagcagcaactgctaaagctgtagaagcagcaacttcatcagcttctgcttcatctagtcaagctccacaagtaagtacaagcactgataatacaacatcaaatgctagtgcctcaaacagttctaatagttcatcaaactcaagttcaagttctagcagttcatcaagctcaagctcaagctcaagtaattctaatgctggtgggaatacaaattcaggcactagtactggaaatactggaggaacaactactggtggtagcggtataaatagttcaccaattggaaatccttatgctggtggtggatgtactgactatgtatggcaatactttgctgcacaaggaatttatatcagaaatatcatgcctggtaatggtggacaatgggcttctaatggacctgcccaaggcgtgctccatgttgtaggagctgctcctggtgttatcgcatcaagcttctcagctgattttgttggatatgcaaactcaccttacggtcacgtagctattgtaaaatcagttaattcagatggtacaattactatcaaagaaggcggatatggtacaacttggtggggacatgaacgtactgtaagtgcgtctggtgttactttcttgatgccaaactagaaaaaagtcttaataaataaaaaatagtggtttgatagtggggaataattttccttctgtcaaatcattttttattattgtggtataataataaggaaaaatgata

SEQ ID NO: 13 - protein, 292 aa, Bacillus subtilis
MKKIAIAAITATSILALSACSSGDKEVIAKTDAGDVTKGELYTNMKKTAGASVLTQLVQEKVLDKKYKVSDKEIDNKLKEYKTQLGDQYTALEKQYGKDYLKEQVKYELLTQKAAKDNIKVTDADIKEYWEGLKGKIRASHILVADKKTAEEVEKKLKKGEKFEDLAKEYSTDSSASKGGDLGWFAKEGQMDETFSKAAFKLKTGEVSDPVKTQYGYHIIKKTEERGKYDDMKKELKSEVLEQKLNDNAAVQEAVQKVMKKADIEVKDKDLKDTFNTSSTSNSTSSSSSNSK

SEQ ID NO: 14 - protein, 719 aa, Aspergillus niger
MNTLLIHHWTALNNALSSNDIGRDLDGKTLDLATVVASARYGKRVSLSQSTRKHVQRSEAVLRKHLADGELIYGVNTGFGGSADTTTDAVNKLQENLLQMLQAGVKLDGSSPVVVNKPNEDILPLDDPISATIMPESWTRATMIIRLNCLASGSSGIRQSVLDVLLRMIEQHITPRVPLRGSISASGDLSPLSYIGGAMQGHPSIHVWKRGRNSDCQVKGADVALKESSIPFVCLDAKEGLAIVNGTATSAAVGGLALHETLGLAALSQVLAAMSVEALHGTSESFDPFFAKVRPHPGQTTAANNLYKFLSNSKLLNDGNDFRQGSLRQDRYSIRTSAQWMGPLLEDLELAYQQISVELNSVTDNPLIDTAGDNARILHGGNFQAKSITSAMEKTRQAVQSMGRMLYAQCTELIDSSKNNGLPPNLSADEPSLSFTFKGVDIMIAALQSELGFLANPIGSHVQTAEMGNQALNSLALISSRYTLDAVAVLSQMAAAHLVATCQALDLRVINIQFMRTLEQDFMKLFGDSFQVNAVPFKNILAISKEAWDAFAKQVEQLQSVDSCSRFHQAARAILPIFIMALPQSAASPAIIQSWIDRLGSQATETYRKTHEAYCKCPDATPYLGEASRKMYTHVRQNLKLPFIVEGTLWRPSDSAKTPDGEATPPPLPTMGDVVGIAYDAIRRGSLYTVAVECIRDVEQDMGKYPHCLVNYPTCASRT

SEQ ID NO: 15 - protein, 690 aa, Puccinia graminis
MAHADLCSAISRELEEHISNKREIILDGHGLTSTGVVGAARYNVQAKISNDPALITVVEESVEFLASKLDTAIYGVTTFSERLDPRPTNACNFYFQGSADTRSDSTADLQMAFLEHQVSGVLPLSSRSTSAGLYLSDPMNNVMPEAITRGAILIRINSLVRGHSALRFQVLETLITLLNKNLTPMVPLRGSISASGDLMPLSYVAAAICGHPAIRIIDRSSADGHIEIMPASDALTKHGITPIVLGPKEGLAISNGTAFSASAACLVAHDSHMLLMLAQGLTSMTVEAMMGQAQSFDPFIHETCRPHPGQVEVAKTIRSMFEGSRLVIHMDEERSVDQEKDQGILRQDRYALRTAPQWLGPQLEELVTVNKTLCREINATTDNPLIDIKNKKILNGGNFQAMSITNSMEKTRSSLESIGKLSFAQAIELMNCTMSKGLPSCLAGDEPSTNYHTKGLDINMAAYTAELGFLASPVSTHVQSAEQHNQSVNSLALVSARYTIQAVEVLSMLLSSHLYVVCMAIDLRVIDQMFQKELKGLLPVLLDSHFKSRPTQAADPLIGALASRLEATASLDSEARFLSAFKQTLHVILAFPVDLEEARSWPSFAASQSTLLYKRTRDQYFENSESFLAEKWLGKKNKHLYHFVRKELGIGPRRGDVRLGRHEGSVSIDVSKIYESVRSGELYKFMNRMF

SEQ ID NO: 16 - protein, 33 aa, Lactococcus lactis
TSISLCTPGCKTGALMGCNMKTATCHCSIHVSK

SEQ ID NO: 17 - protein, 9 aa, artificial
  amino acid sequence of synthetic peptide for enhancing secretion
LEISSTCDA

(19) United States
(12) Patent Application Publication
(10) Pub. No.: US 2015/0246085 A1
(43) Pub. Date: Sep. 3, 2015

(54) Genetically-Modified Probiotic for Treatment of Phenylketonuria

(71) Applicant: THE SYDNEY CHILDREN'S HOSPITAL NETWORK (RANDWICK & WESTMEAD), Westmead, New South Wales (AU)

(72) Inventors: Naz Al-Hafid, New South Wales (AU); John Christodoulou, New South Wales (AU); Xing Zhang Tong, New South Wales (AU)

(21) Appl. No.: 14/440,135

(22) PCT Filed: Nov. 1, 2013
(86) PCT No.: PCT/AU2013/001262

§ 371 Date: May 1, 2015

(30) Foreign Application Priority Data
Nov. 1, 2012 (AU) 2012904813

(51) Int. Cl. A61K 35/747; C12N 9/88; A61K 38/51; A61K 35/744

(52) U.S. Cl. CPC A61K 35/747; A61K 35/744; C12N 9/88; A61K 38/51; C12Y 403/01024; A61K 2035/115

(57) ABSTRACT

The present disclosure provides a genetically-modified probiotic expressing recombinent phenylalanine ammonia Lyase (PAL) useful for treating phenylketonuria.

### PRIORITY DETAILS

The present application claims priority from Australian Provisional Patent Application No. 2012904813 entitled “Modified microorganism and uses thereof” filed on 1 Nov. 2012, the entire contents of which are hereby incorporated by reference.

### SEQUENCE LISTING

The present application is accompanied by a Sequence Listing filed in electronic form, the entire contents of which are hereby incorporated by reference.

### FIELD

The present disclosure relates to a genetically-modified probiotic useful for treating, e.g., phenylketonuria.

### BACKGROUND

Phenylketonuria (PKU) is an autosomal recessive genetic disorder, caused by mutations in the phenylalanine hydroxylase (PAH) gene (expressed primarily in the liver), and which leads to an abnormality in phenylalanine metabolism, resulting in hyperphenylalaninaemia (HPA) and potentially very severe intellectual disability. Dietary management from birth with a low phenylalanine diet largely prevents the development of the neurological consequences of the disorder, and is recognized as the most effective treatment that is currently available for the majority of affected individuals. However, adherence to life-long dietary treatment is difficult, particularly beyond school age. Over the last few years, several treatment strategies have emerged, including improving the palatability of the available medical foods, large neutral amino acid supplementation, provision of pharmacological doses of the cofactor tetrahydrobiopterin, enzyme replacement and enzyme substitution. However, these proposed strategies suffer from shortcomings.

For example, life-long dietary treatment incurs a significant expense to the subject suffering from PKU and their family The cofactor tetrahydrobiopterin can only be used in some mild forms of PKU. As for enzyme replacement, such treatment requires a large intact multi-enzyme complex and PAH is renowned for being unstable.

An enzyme that maybe used to substitute for PAH is phenylalanine ammonia-lyase (PAL). PAL efficiently converts phenylalanine to metabolically insignificant amounts of ammonia and transcinnamic acid, a harmless metabolite. Some reports have shown that PAL can effectively control phenylalanine blood levels in animal models for PKU by either intravenous or oral administration. However, intravenous PAL, being a foreign protein, can cause an immune response that can neutralize PAL and/or be dangerous to the subject. As for oral administration, preliminary studies of this strategy were not continued because PAL was not available in sufficient amounts at a reasonable cost to permit a longer-term study. Oral administration of E. coli expressing recombinant PAL has also been considered, however this bacteria has the potential to cause gastroenteritis in human, and is undesirable for therapeutic applications in humans.

It will be apparent from the foregoing discussion, that therapeutics for PKU are desirable.

### SUMMARY

In producing examples of the present disclosure, the inventors produced genetically-modified probiotics, e.g., bacteria, expressing phenylalanine ammonia lyase (PAL). These genetically-modified probiotics were shown to be able to convert phenylalanine to metabolically insignificant amounts of ammonia and trans-cinnamic acid, even when the PAL was expressed intracellularly. The probiotic was shown to be able to metabolise free phenylalanine or phenylalanine contained within a polypeptide. The inventors showed that the probiotic was able to survive following exposure to conditions mimicking various compartments of the human gastro-intestinal tract. The inventors also showed that by administering the probiotic they could reduce increases in levels of phenylalanine in blood from wild-type mice and from a mouse model of PKU following administration of the amino acid. These findings by the inventors provide the basis for reagents and methods for treating PKU or preventing its consequences.

In one general example, the present disclosure provides a genetically-modified probiotic expressing recombinant PAL. For example, the probiotic expresses PAL at a level such that when a composition comprising a plurality of the probiotics is administered to a subject with phenylalanine, the level of phenylalanine in the blood of the subject is significantly lower than the level observed in a subject to whom phenylalanine has been administered in the absence of the composition. For example, in a subject suffering from PKU, the level of increase of phenylalanine in blood is at least 25% lower or 30% lower or 40% lower or 50% lower or 60% lower or 70% lower or 80% lower than the level observed in a subject to whom phenylalanine has been administered in the absence of the composition.

In one example, the phenylalanine administered with the probiotic is labelled phenylalanine (e.g., isotope labelled, such as labelled with deuterium). In one example, the probiotic expresses PAL at a level sufficient to metabolize phenylalanine at a level that is within about 2.5% or 3.5% or 6.5% or 20% or 24% of the level at which PAL from Rhodotorula glutinis metabolizes phenylalanine. For example, a culture of probiotic (e.g., a culture of bacterial probiotic having an OD₆₀₀ of about 0.4-0.6) is lysed and contacted (e.g., 20 μl of lysate is contacted) to a composition comprising 10 mmol phenylalanine (e.g., about 1 ml of the composition, in Tris buffer) and the amount of transcinnamic acid formed is determined. The amount of transcinnamic acid formed is compared to the amount formed in the presence of 2 μg of PAL from R. glutinis.

In one example, the probiotic remains viable following exposure to a gastric and/or gastrointestinal tract of a subject or to an environment mimicking one or more conditions of a compartment of a gastric and/or gastrointestinal tract of a subject. For example, the probiotic remains viable following exposure to one or more or all of the following conditions:
pH5.0 in the presence of 0.01% lysozyme and 0.3% pepsin—mimicking the upper stomach of a human;
pH4.1—mimicking the middle stomach of a human;
pH3.0—mimicking the middle stomach of a human;
pH2.1—mimicking the lower stomach of a human;
pH6.5—mimicking the proximal duodenum of a human; and/or
pH8.0 in the presence of 0.45% bile salts and 0.1% pancreatin—mimicking the duodenum of a human.

In one example, the probiotic is a bacterium, for example, a Gram-positive bacterium. For example, the probiotic is a lactic acid bacterium, such as a bacterium of the genus Lactococcus, Lactobacillus, Leunostoc, Pediococcus and Strepococcus Spp.

In one example, the probiotic is of the genus Lactobacillus.

In one example, the probiotic is of the genus Lactococcus. For example, the probiotic is L. lactis. For example, the L. lactis (or other probiotic) expresses the genes nisK and nisR. Such expression can be a result of genetic modification.

In one example, the probiotic is generally recognised as safe.

Thus, in one example, the present disclosure provides a genetically modified L. lactis expressing recombinant PAL. Exemplary strains of L. lactis useful in the present disclosure include IL1403, NZ9700, NZ9800, NZ9000, NZ3900 and NZ3000. In one example, the strain is NZ9000. In one example, the strain is IL1403.

In one example, the PAL is from a plant or a fungus. For example, the PAL is from a plant, for example a dicotyledonous plant. For example, the PAL is from a plant of the family Apiaceae, for example of the genus Petroselinum. In one example, the PAL is from P. crispum (parsley).

Thus, in one example, the present disclosure provides a genetically modified L. lactis expressing recombinant PAL from P. crispum (parsley).

In one example, nucleic acid encoding the PAL is operably linked to a promoter an expression construct in the probiotic. The expression construct may be integrated into the genome of the probiotic or may remain episomal, e.g., as an expression vector.

Thus, in one example, the present disclosure provides a genetically modified L. lactis or Lactobacillus expressing recombinant PAL from P. crispum (parsley), wherein the nucleic acid is operably linked to a promoter in an expression construct in the L. lactis or Lactobacillus.

In one example, the promoter is a constitutive promoter operable in L. lactis or Lactobacillus.

In one example, the promoter is an inducible promoter operable in L. lactis or Lactobacillus. For example, the promoter is inducible in response to the presence of nisin. For example, the promoter is a nisA promoter or functional fragment thereof.

Thus, in one example, the present disclosure provides a genetically modified L. lactis or Lactobacillus expressing recombinant PAL from P. crispum (parsley), wherein nucleic acid encoding the PAL is codon optimized for expression in the L. lactis or Lactobacillus, and wherein the nucleic acid is operably linked to a nisA promoter or functional fragment thereof.

In one example, the nucleic acid encoding the PAL is codon optimized for expression in the probiotic. For example, the PAL is from P. crispum (parsley) and the encoding nucleic acid is codon optimized for expression in L. lactis or Lactobacillus. An exemplary nucleic acid comprises the sequence set forth in SEQ ID NO: 1.

Thus, in one example, the present disclosure provides a genetically modified L. lactis or Lactobacillus expressing recombinant PAL from P. crispum (parsley), wherein nucleic acid encoding the PAL is codon optimized for expression in the L. lactis, and wherein the nucleic acid is operably linked to a promoter in an expression construct in the L. lactis or Lactobacillus.

The present disclosure also provides a genetically modified L. lactis or Lactobacillus expressing recombinant PAL from P. crispum (parsley), wherein nucleic acid encoding the PAL is codon optimized for expression in the L. lactis or Lactobacillus, and wherein the nucleic acid is operably linked to a nisA promoter or functional fragment thereof.

The present disclosure also provides a genetically modified L. lactis or Lactobacillus expressing recombinant PAL from P. crispum (parsley), wherein nucleic acid encoding the PAL is codon optimized for expression in the L. lactis or Lactobacillus, and wherein the nucleic acid is operably linked to a nisA promoter or functional fragment thereof, and wherein the L. lactis or Lactobacillus expresses PAL at a level sufficient to metabolize phenylalanine at a level that is within about 24% of the level at which PAL from Rhodotorula glutinis metabolizes phenylalanine.

In one example, the PAL is expressed intracellularly. The present inventors have shown that despite the PAL being expressed intracellularly, the probiotic is able to metabolize significant levels of phenylalanine.

In another example the PAL is secreted. For example, the PAL is expressed as a fusion protein with a secretion signal operable in the probiotic. Exemplary secretion signals operable in Lactococcus include the secretion signal from USP45 (optionally wherein the sequence LEIS STCDA (SEQ ID NO: 17) is included between the secretion signal and the PAL), or the secretion signal from Lactobacillus brevis S-layer protein.

In another example, the probiotic additionally expresses a chaperone to thereby increase expression of the PAL. For example, the probiotic is genetically modified to express the chaperone. An exemplary chaperone is Bacillus subtilis chaperone-like protein PrsA.

In a further example, the probiotic additionally expresses a protein that confers resistance to bile salts, e.g., a bile salt hydrolase. For example, the probiotic additionally expresses bilE, e.g., from Listeria monocytogenes. In one example, the probiotic is genetically modified to express the enzyme.

In a still further example, the probiotic is encapsulated, e.g., in a composition that confers resistance to the gastrointestinal tract. In one example, the probiotic is microencapsulated.

The present disclosure additionally provides a cell line, the cell line comprising a clonal population of the probiotic of the disclosure.

The present disclosure additionally provides a cell bank comprising a probiotic of the disclosure. In one example, the cell bank is a frozen cell bank. For example, the cell bank is a master cell bank (e.g., comprising samples of the probiotic for long term storage) or a working cell bank (e.g., comprising samples of the probiotic for manufacturing, e.g., expansion prior to formulations and/or administration).

The present disclosure additionally provides a composition comprising the probiotic of the disclosure or an encapsulated form thereof and a carrier.

In one example, the composition is a foodstuff, e.g., a solid or liquid foodstuff. In this case, the carrier can comprise a dairy product, e.g., milk or yoghurt.

In one example, the foodstuff carrier is semi solid, e.g., is gelatinous. In one example, the gelatinous carrier is a jelly, e.g., a flavoured jelly, such as a strawberry flavoured jelly.

In another example, the composition is a pharmaceutical composition and the carrier is pharmaceutically acceptable.

The present disclosure additionally provides a method for reducing levels of phenylalanine in a subject having PKU or preventing an increase in levels of phenylalanine in a subject having PKU after consuming a phenylalanine-containing foodstuff, the method comprising administering to the subject a probiotic of the disclosure or an encapsulated form thereof or a composition of the disclosure.

The present disclosure additionally provides a method for reducing levels of phenylalanine in a subject having PKU or preventing an increase in levels of phenylalanine in a subject having PKU to levels sufficient to prevent the subject developing mental retardation resulting from chronic exposure to increased levels of phenylalanine or to reduce the risk of the mental retardation, the method comprising administering to the subject a probiotic of the disclosure or an encapsulated form thereof or a composition of the disclosure.

In one example, the method comprises administering the probiotic, encapsulated form thereof or composition a sufficient number of times to reduce levels of phenylalanine in a subject having PKU or prevent an increase in levels of phenylalanine in a subject having PKU over time despite the subject consuming phenylalanine-containing food.

The present disclosure additionally provides a method for treating or preventing a symptom of PKU in a subject or for preventing the effects of PKU in a subject (e.g., mental retardation resulting from chronic exposure to increased levels of phenylalanine in a subject), the method comprising administering to the subject a probiotic of the disclosure or an encapsulated form thereof or a composition of the disclosure.

In one example, the probiotic, encapsulated form or composition is administered with food or within three hours or two hours or one or of consuming food.

In one example, the food comprises phenylalanine.

In one example, the probiotic, encapsulated form or composition is administered in an effective amount or a therapeutically effective amount or a prophylactically effective amount.

In one example, the method comprises administering the probiotic, encapsulated form thereof or composition in an amount of at least about 10⁴ to about 10¹⁰ cfu per dose; or about 10⁵ to about 10⁹ cfu per dose; or about 10⁵ to about 10⁷ cfu per dose; or about 10⁹ cfu per dose.

In one example, the method of the disclosure additionally comprising monitoring the level of phenylalanine in a subject (e.g., in the blood of a subject) and administering the probiotic, encapsulated form thereof or composition if the level of phenylalanine exceeds a threshold. For example, the threshold is 300 μmol/L or 320 μmol/L or 340 μmol/L or 350 μmol/L or 360 μmol/L.

The present disclosure also provides a probiotic of the disclosure or an encapsulated form thereof or a composition of the disclosure for use in reducing levels of phenylalanine in a subject having PKU or preventing an increase in levels of phenylalanine in a subject having PKU after consuming a phenylalanine-containing foodstuff or treating or preventing a symptom of PKU.

The present disclosure additionally provides for use of a probiotic of the disclosure or an encapsulated form thereof or a composition of the disclosure in the manufacture of a medicament for reducing levels of phenylalanine in a subject having PKU or preventing an increase in levels of phenylalanine in a subject having PKU after consuming a phenylalanine-containing foodstuff or treating or preventing a symptom of PKU.

The present disclosure also provides an article of manufacture comprising a probiotic of the disclosure or an encapsulated form thereof or a composition of the disclosure.

### BRIEF DESCRIPTION OF THE DRAWINGS

FIG. 1 is a schematic representation of construction of the plasmids pCMK, pMCMK, and pSMC2.

(1). The Nuc coding sequence in pCYT:Nuc was replaced by the PAL coding sequence as an NsiI/HindIII fragment in pMK to generate the vector pCMK. The nisA promoter and the codon-optimised PAL coding sequence from pCMK were cloned into the pMSP3535H3 vector as a BglII/XhoI fragment, yielding the pMCMK vector.
(2). The codon-optimised PAL sequence in pMK was cloned into plasmid pNZ8148 as an NcoI/HindIII fragment to generate the vector pNZ8148MK. A BglII/BstXI fragment containing the nisA promoter and a part of the PAL coding sequence was then excised from p8148MK and cloned into the pMCMK vector cut by BglII and BstXI to generate the pSMC2 vector.

FIG. 2 is a graphical representation of the extraction efficiency of transcinnamate comparing ethyl acetate and Tris-HCl buffer.

FIG. 3 is a schematic representation of PAL activity assay for all constructs. NZ9000: L. lactis NZ9000 is the host strain for all expression constructs, used as a negative control. pCYT:Nuc: expresses staphylococcal nuclease under the control of the nisin inducible promoter PnisA, used as a negative control. pCYT-PAL10: expresses P. crispum PAL under the control of the promoter PnisA in pCYT. pCMK: expresses codon-optimised P. crispum PAL under the control of the promoter PnisA in pCYT. pMCMK: expresses codon-optimised P. crispum PAL under the control of the promoter PnisA in the vector pMSP3535H3. pSMC2: expresses codon-optimised P. crispum PAL under the control of the promoter PnisA in the vector pMSP3535H3. Pure PAL: the purified phenylalanine ammonia-lyase from a yeast Rhodotorula glutinis (Sigma).

FIG. 4 is a graphical representation of the protein gel showing Coomassie staining of cell lysates. Lane 1: Novex protein standard (Invitrogen). Lane 2: L. lactis NZ9000. Lane 3: pSMC2, expresses the codon-optimised P. crispum PAL under the control of the promoter PnisA in the vector pMSP3535H3. Lane 4: pSMC5, expresses the codon-optimised P. crispum PAL under the control of the promoter PnisA in the vector pMSP3535H3. Lane 5: purified phenylalanine ammonia-lyase from a yeast Rhodotorula glutinis (Sigma).

FIG. 5 is a graphical representation of Standard curve of the optical density (600 nm) of the genetically-modified probiotic and their corresponding number of cells/ml (log 10).

FIG. 6 is a graphical representation of the reaction rate of PAL at the different temperatures in intact probiotic bacteria, as measured by the rate of clearance of the labelled phe (d8Phe).

FIG. 7 is a graphical representation of the transcinnamate production by the PAL expressing probiotic. SMC2 Un-ind=probiotic grown in culture broth but not induced with nisin; SMC2=probiotic induced with nisin and cultured in standard M17 broth; SMC2+phe=probiotic induced with nisin and cultured in M17 broth “spiked” with free L-phenylalanine.

FIG. 8 is a graphical representation of wildtype mice treatment with probiotic and labelled phenylalanine.

FIG. 9 is a graphical representation of PKU mice treatment with probiotic and labelled phenylalanine (p<0.05; Mann-Whitney unpaired test).

FIG. 10 is a diagrammatic representation showing an alignment of PAL sequences from various plant species as indicated.

FIG. 11 is a diagrammatic representation showing an alignment of PAL sequences from two fungal species as indicated.

### KEY TO SEQUENCE LISTING

SEQ ID NO 1: nucleotide sequence encoding Petroselinum crispum codon-optimised Phenylalanine ammonia lyase (PAL)
SEQ ID NO: 2 amino acid sequence of Petroselinum crispum PAL
SEQ ID NO: 3 amino acid sequence of Daucus carota PAL
SEQ ID NO: 4 amino acid sequence of Catharanthus roseus PAL
SEQ ID NO: 5 amino acid sequence of Rudbeckia hirta PAL
SEQ ID NO: 6 amino acid sequence of Lactuca sativa PAL
SEQ ID NO: 7 amino acid sequence of Listeria monocytogenes bile salt hydrolase (bilE)
SEQ ID NO: 8 nucleotide sequence encoding Listeria monocytogenes bile salt hydrolase (bilE)
SEQ ID NO: 9 amino acid sequence encoding Lactobacillus brevis S-layer protein
SEQ ID NO: 10 nucleotide sequence encoding Lactobacillus brevis S-layer protein
SEQ ID NO: 11 amino acid sequence of Lactococcus lactis USP45 signal peptide
SEQ ID NO: 12 nucleotide sequence encoding Lactococcus lactis USP45 signal peptide
SEQ ID NO: 13 amino acid sequence of Bacillus subtilis chaperone-like protein PrsA
SEQ ID NO: 14 amino acid sequence of Aspergillus niger PAL
SEQ ID NO: 15 amino acid sequence of Puccinia graminis PAL
SEQ ID NO: 16 amino acid sequence of Lactococcus lactis nisin-A
SEQ ID NO: 17 amino acid sequence of synthetic peptide for enhancing secretion

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally equivalent products, compositions and methods are clearly within the scope of the disclosure.

The present disclosure is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in Sambrook, Fritsch & Maniatis, Molecular Cloning: A Laboratory Manual, Cold Spring Harbor Laboratories, New York, Second Edition (1989), whole of VoIs I, II, and DI; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp1-22; Atkinson et al, pp35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Wünsch, E., ed. (1974) Synthese von Peptiden in Houben-Weyls Metoden der Organischen Chemie (Miller, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000), ISBN 0199637970, whole of text.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “consisting essentially of” will be understood to limit the scope of a claim to the specified compositions or steps while still encompassing those that do not materially affect the basic and characteristic(s) of the composition or method.

As used herein, the term “consisting of” excludes any element, step, or ingredient not specified in the claim except for impurities ordinarily associated therewith.

### Selected Definitions

As used herein, the term “probiotic” will be taken to mean a live microorganism which when administered in adequate amounts confer a health benefit on a subject. Health benefits are a result of, for example, production of nutrients and/or co-factors by the probiotic, competition of the probiotic with pathogens and/or stimulation of an immune response in the subject by the probiotic. Exemplary probiotics are generally recognized as safe (GRAS).

As used herein, the term “generally recognized as safe” or “GRAS” refers to prokaryotic or eukaryotic microorganisms that, based on experimental data and practical use experience, have been found not to produce substantial levels of toxic or otherwise hazardous substances or to have adverse effects when ingested by higher organisms including humans and other mammals. A listing of exemplary microorganisms generally recognised as safe is available in the GRAS Notice Inventory at the US Food and Drug Administration. The group of GRAS organisms includes microorganisms that are conventionally used in the manufacturing of food products. Typical examples of such organism are the group of lactic acid bacteria that are used as starter cultures in the dairy industry, the feed industry and other industries concerned with the manufacturing of product where lactic acid bacterial cultures are used. This term also encompasses obligate anaerobic bacteria belonging to the Bifidobacterium genus which are taxonomically different from the group of lactic acid bacteria. Other examples of GRAS organisms are yeast species used in food manufacturing such as baker's yeast, brewer's yeast and yeast organisms used in the fermentation of wine and other beverages. Typical examples of yeast species that can be considered as GRAS organisms include Saccharomyces cerevisiae and Schizosaccharomyces pombe. The use of filamentous fungi having GRAS status is also contemplated.

As used herein, the term “phenylalanine ammonia lyase” or “PAL” is a large group of proteins expressed in, for example, plants and fungi that catalyse nonoxidative deamination of L-phenylalanine to form trans-cinnamic acid and a free ammonium ion. Exemplary PALs are described in Hyun et al., Mycobiology, 39: 257-265, 2011. Some exemplary PALs are set out in SEQ ID Nos: 2-6, 14 and 15 and FIGS. 10 and 11.

As used herein, the term “lactic acid bacterium” designates a bacterium of the group of Gram positive, catalase negative, non-motile, microaerophilic or anaerobic bacteria which ferment sugar with the production of acids including lactic acid as the predominantly produced acid, acetic acid, formic acid and propionic acid. Exemplary lactic acid bacteria are found among Lactococcus species including Lactococcus lactis, Streptococcus species, Enterococcus species, Lactobacillus species, Leuconostoc species, Oenococcus species and Pediococcus species.

Reference herein to a probiotic “remaining viable following exposure” to a condition means that after exposing a population of the probiotic to a condition it is possible to culture the probiotic. This does not mean that every probiotic cell that is exposed to the condition remains viable, only that following exposure to the condition detectable numbers of the probiotic can be cultured.

As used herein, the term “genetically modified” will be understood to mean that a probiotic has undergone modification to introduce a nucleic acid that does not naturally occur in the probiotic or to introduce additional copies or modified forms of nucleic acids that naturally occur in the probiotic. The nucleic acid can be integrated in one or more copies into a genome of the probiotic or one or more copies of the nucleic acid can remain episomal, e.g., in a plasmid, phagemid or artificial chromosome. This term does not require active modification of each and every probiotic of the disclosure. Rather a probiotic can be genetically modified and a population of probiotics generated therefrom, e.g., by standard culturing methods, will also be considered to be “genetically modified”.

As used herein, the term “recombinant” in the context of a protein or polypeptide (e.g., PAL) will be understood to mean a protein that is expressed as a result of a genetic modification of a probiotic, e.g., a protein or polypeptide encoded by a nucleic acid introduced into a probiotic by genetic modification.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked” means positioning a promoter relative to a nucleic acid such that expression of the nucleic acid is controlled by the promoter.

As used herein, the term “constitutive promoter” will be understood to mean a promoter that directs expression of a nucleic acid to which it is operably linked for the most part, or entirely, independent of environmental and developmental factors. As their expression is normally not conditioned by endogenous factors, constitutive promoters are usually active across species.

As used herein, the term “inducible promoter” will be understood to mean a promoter that directs expression of a nucleic acid to which it is operably linked in response to an environmental stimulus, such as a compound, light, oxygen levels, heat or cold.

As used herein, the term “expression construct” refers to a nucleic acid that has the ability to confer expression on a nucleic acid to which it is operably connected, in a cell. Within the context of the present disclosure, it is to be understood that an expression construct may be an expression vector or a linear nucleic acid (e.g., DNA), which can integrate into a genome of a probiotic.

As used herein, the term “expression vector” will be understood to mean a nucleic acid comprising an expression construct and that is capable of maintaining and/or replicating DNA in an expressible format. Exemplary expression vectors include plasmids, bacteriophage, phagemids, cosmids, virus sub-genomic or genomic fragments and artificial chromosomes.

As used herein, the term “nisin” will be understood to refer to a 34-amino acid anti-microbial peptide (lantibiotic) with various unusual amino acids and five ring structures, e.g., as described in Mierau and Kleerebezem Appl. Micriobiol. Biotechnol., 68: 705-717, 2005. An exemplary sequence of nisin from L. lactis is set forth in SEQ ID NO: 16.

As used herein, the term “codon optimized” will be understood to mean that a sequence of a nucleic acid encoding a protein or polypeptide is produced that includes codons that are preferentially used by the organism in which the polypeptide or protein is to be expressed. Methods for determining which codons are preferentially used in an organism may be established by standard means, e.g., by reference to a published codon preference for the organism(s) in question and/or are known in the art and described, for example, in Wan et al., BMC Evolutionary Biology 4, 19, 2004; Chen et al., Proc. Nat. Acad. Sci. USA 101, 3480-3485, 2004, McLachlan et al., Nucleic Acids Res. 12(24), 9567-9575, 1984, Ikemura J. Mol. Biol. 146(1), 1-21, 1981; Ikemura J. Mol. Biol. 158(4), 573-97, 1982, Bennetzen & Hall, J. Biol. Chem. 257(6), 3026-3031, 1982, Gribskov et al., Nucleic Acids Res. 12(1), 539-549, 1984, Sharp and Li Nucleic Acids Res. 15(3), 1281-1295, 1987, Stenico et al., Nucleic Acids Res. 22: 2737-2446, 1993, and Fuglsan APMIS 111: 843-842, 2003. Codon usage information is also available for a variety of organisms from the “Codon Usage Database” available from Kazusa DNA Research Institute.

As used herein, the term “secretion signal” will be understood to mean a region of a polypeptide that effects secretion of a polypeptide to which it is linked across the cell membrane (and/or cell wall) in a cell in which it is expressed.

As used herein, the term “encapsulate” will be understood to mean that a probiotic or a plurality of probiotics of the disclosure are coated in a composition. In one example, the probiotic is encapsulated in a composition that protects the probiotic from gastric conditions and, for example, that releases the probiotic in the intestine, such as the small intestine, of a subject. Exemplary encapsulants are described herein.

As used herein, “treatment” or “treating” is an approach for obtaining beneficial or desired results including clinical results. For purposes of this disclosure, beneficial or desired clinical results include, but are not limited to, one or more of the following: decreasing one or more symptoms resulting from a condition, diminishing the extent of the condition, stabilizing the condition (e.g., preventing or delaying the worsening of the condition), delay or slowing the progression of the condition, ameliorating the condition, decreasing the dose of one or more other medications required to treat the condition, and/or increasing the quality of life.

As used herein, “delaying” the progression of a condition means to defer, hinder, slow, retard, stabilize, and/or postpone development of the condition. This delay can be of varying lengths of time, depending on the history of the condition and/or individual being treated.

As used herein, the term “prevent” or “preventing” or “prevention” shall be taken to mean stopping or hindering or reducing the development of at least one symptom of a clinical condition.

### Probiotics

From the description herein, the skilled person will be able to identify suitable probiotics for use in the present disclosure.

In one example, the probiotic is a fungus. Exemplary fungal probiotics are of the genus Saccharomyces or Schizosaccharomyces. For example, a fungal probiotic is Saccharomyces cerevisiae, Saccharomyces boulardii or Schizosaccharomyces pombe.

In an exemplary form of the disclosure, the probiotic is a bacterium, such as Lactococcus lactis, Lactobacillus acidophilus, L. rhamnosus, L. casei, L. Reuteri, L. amylovorus, L. brevis, L. delbrueckii, L. gallinarum, L. johnsonii, L. plantarum, L. salivarius, L. ansporogenes, Bifidobacterium bifidum, B. animalis, Streptococcus thennophilus, S. cremoris, S. faecium, S. infantis or Enterococcus faecium.

In one example, the probiotic is not Escherichia coli.

In one example, the probiotic is of the genus Lactobacillus. In one example, the probiotic is L. acidophilus. In another example the probiotic is L. casei. In a further example the probiotic is L. plantarum.

In one example, the probiotic is of the genus Lactococcus. In one example, the probiotic is L. lactis. Exemplary strains of L. lactis suitable for performance of the present disclosure are described herein.

In one example, the probiotic is one that is readily genetically-modified.

In one example, the probiotic is one in which a nisin-inducible promoter (e.g., nisA) is operable. In accordance with this example, the probiotic may be genetically modified to express nisK and nisR.

Two common groups of L. lactis hosts have been produced: L. lactis strains that can produce nisin, such as L. lactis FI5876 and L. lactis NZ9700, which were from wild-type strains and cured of plasmids and prophage; and strains that can not produce nisin.

The first group was derived from nisin-producing strains, such as L. lactis NZ9800 from L. lactis NZ9700, with a 4 bp gene deletion in nisA, FI7332 from FI5876, with an Emr gene integrated into the Sac′ site of the nisA gene, and L. lactis FI7847 from L. lactis FI5876, with a 20 bp insertion in nisA. The second type was from non-nisin-producing L. lactis or other genera bacteria, with a nisRK gene integrated into the chromosome, such as L. lactis NZ9000 and L. lactis NZ3900 (also with a lacF deletion to be used for foodgrade selection).

Presently, a commonly used host strain is NZ9000. To construct this strain the genes for nisK and nisR were integrated into the pepN gene of MG1363. The two genes are transcribed from their own constitutive promoter.

Strain NZ3900 was developed for food-grade applications of the NICE system. It is derived from strain NZ3000, which is a lacF deletion mutant of strain MG5267, a strain with a single chromosomal copy of the lactose operon of the dairy starter strain NCDO712. The lactose operon was transferred to strain MG1363 bp transduction, creating strain MG5267. Due to the lacF deletion, strain NZ3000 is unable to grow on lactose. However, growth on lactose can be restored by providing lacF on a plasmid, thus permitting selection of genetically modified bacteria.

### PAL

As discussed above, PALs have been identified in or isolated from a large number of plant and fungal species. The sequences of PALs are available from publicly available resources, such as the database of the National Center of Biological Information (NCBI).

Exemplary sequences of PAL are also described herein. For example, sequences of exemplary plant PALs are shown in FIG. 10. Sequences of exemplary fungal PALs are shown in FIG. 11.

In one example, the PAL is from parsley. For example, the PAL is from a leaf parsley or a root parsley.

In one example, a PAL has a sequence that is at least about 80% identical to the sequence set forth in SEQ ID NO: 2. In one example, the percentage identity is at least about 85% or 90% or 95% or 96% or 97% or 98% or 99% or 100%. In this regard, FIG. 10 herein shows the sequences of PALs from plants having at least 85% or 86% or 95% identity thus indicating positions in which mutations can be made to a sequence without disrupting protein function.

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

In one example, a PAL comprises one or more (e.g., 2 or 3 or 4 or 5 or 6 or 7 or 8 or 9 or 10 or 11 or 12 or 13 or 1 or 15 or 16 or 17 or 18 or 19 or 20) conservative amino acid changes relative to SEQ ID NO: 2. A “conservative amino acid substitution” is one in which the amino acid residue is replaced with an amino acid residue having a similar side chain.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The importance of the hydropathic amino acid index in conferring interactive biological function on a protein is also generally understood in the art (Kyte & Doolittle, J. Mol. Biol. 157, 105-132, 1982). It is known that certain amino acids may be substituted for other amino acids having a similar hydropathic index or score and still retain a similar biological activity, for example, the ability to induce membrane repair. The hydropathic index of amino acids also may be considered in determining a conservative substitution that produces a functionally equivalent molecule.

It is also understood in the art that the substitution of like amino acids is made effectively on the basis of hydrophilicity. As detailed in U.S. Pat. No. 4,554,101, the following hydrophilicity values have been assigned to amino acid residues: arginine (+3.0); lysine (+3.0); aspartate (+3.0+/−0.1); glutamate (+3.0+/−0.1); serine (+0.3); asparagine (+0.2); glutamine (+0.2); glycine (0); threonine (−0.4); proline (−0.5+/−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, amino acids having hydrophilicity values within about +/−0.2 of each other, such as within about +/−0.1, for example, within about +/−0.05 are substituted.

In one example, the PAL comprises, consists essentially of or consists of a sequence set forth in SEQ ID NO: 2.

### Signal Sequences

In one example, a PAL is secreted from the probiotic.

In one example, the PAL is expressed as a fusion protein with a signal sequence (also known as a signal peptide) that mediates secretion of the PAL.

Exemplary secretion signals operable in a probiotic, such as Lactococcus, e.g., L. lactis, generally include three distinct regions: (i) an N-terminal region (n-region) that contains a number of positively charged amino acids (e.g., lysines and arginines); (ii) a central hydrophobic core region (h-region); and (iii) a hydrophilic cleavage region (c-region) that contains the sequence motif recognized by the signal peptidase. Exemplary secretion signals include:
Signal Peptide from nuc: this signal peptide is atypical, as it is 60 residues and contains two hydrophobic stretches that may form a hairpin in the cytoplasmic membrane during translocation. Details of the nuc signal peptide are described in Simonen et al., Microbiol Rev. 57:109-113, 1993;
Signal Peptide 310 and mutants thereof, e.g., SP310mut2 as described in Bermudez et al., Infect Immun 71: 1887-1896, 2003; or
Signal Peptide Exp4 as described in Dieye et al., Bacteriol 183: 4157-4166, 2001.

In one example, the secretion signal is from the USP45 protein. For example, the secretion signal comprises a sequence set forth in SEQ ID NO: 11.

In another example, the secretion signal is from the Lactobacillus brevis S-layer protein. For example, the secretion signal comprises a sequence set forth in SEQ ID NO: 9.

In one example, a peptide comprising the sequence LEISSTCDA (SEQ ID NO: 17) is included between the secretion signal and the PAL.

### Additional Polypeptides Expressed by the Probiotic

In one example, the probiotic expresses one or more additional recombinant polypeptides.

For example, the probiotic expresses a polypeptide that facilitates enhanced expression and/or folding and/or secretion of PAL.

For example, the probiotic additionally expresses a chaperone to thereby increase expression of the PAL. For example, the probiotic is genetically modified to express the chaperone. An exemplary chaperone is Bacillus subtilis chaperone-like protein PrsA (e.g., comprising a sequence set forth in SEQ ID NO: 13).

In a further example, the probiotic additionally expresses a polypeptide that confers resistance to bile salts, e.g., a bile salt hydrolase. For example, the probiotic additionally expresses bilE, e.g., from Listeria monocytogenes, e.g., comprising a sequence set forth in SEQ ID NO: 7. In one example, the probiotic is genetically modified to express the polypeptide.

### Recombinant Expression

In one example, a polypeptide (e.g., PAL or other polypeptide described herein) is produced as using recombinant means. To facilitate the production of a recombinant polypeptide, nucleic acid encoding same is isolated or synthesized. Typically the nucleic acid encoding the constituent components of the polypeptide is/are isolated using a known method, such as, for example, amplification (e.g., using PCR or splice overlap extension) or isolated from nucleic acid from an organism using one or more restriction enzymes or isolated from a library of nucleic acids. Methods for such isolation will be apparent to the ordinary skilled artisan and/or described in Ausubel et al (In: Current Protocols in Molecular Biology. Wiley Interscience, ISBN 047 150338, 1987), Sambrook et al (In: Molecular Cloning: Molecular Cloning: A Laboratory Manual, Cold Spring Harbor Laboratories, New York, Third Edition 2001).

Nucleic acid can also be produced synthetically.

In one example, the nucleic acid is provided in the form of an expression construct. As used herein, the term “expression construct” refers to a nucleic acid that has the ability to confer expression on a nucleic acid to which it is operably connected, in a cell. Within the context of the present disclosure, it is to be understood that an expression construct may be an expression vector such as a plasmid, bacteriophage, phagemid, cosmid, virus sub-genomic or genomic fragment, or other nucleic acid capable of maintaining and/or replicating DNA in an expressible format. An expression construct may also be a linear DNA, which integrates into the genome of a cell.

Methods for the construction of a suitable expression construct for performance of the disclosure will be apparent to the skilled artisan and are described, for example, in Ausubel et al (In: Current Protocols in Molecular Biology. Wiley Interscience, ISBN 047 150338, 1987) or Sambrook et al (In: Molecular Cloning: Molecular Cloning: A Laboratory Manual, Cold Spring Harbor Laboratories, New York, Third Edition 2001). For example, each of the components of the expression construct is amplified from a suitable template nucleic acid using, for example, PCR and subsequently cloned into a suitable expression construct, such as for example, a plasmid or a phagemid.

### Promoters

As will be apparent to the skilled person from the foregoing discussion, a nucleic acid encoding a polypeptide (e.g., a PAL or another polypeptide expressed in a probiotic) is operably linked to a promoter that is operable in the probiotic. Suitable promoters will be apparent to the skilled person and/or described herein.

Typical promoters suitable for expression in yeast cells such as for example a yeast cell selected from the group comprising S. cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Promoters operable in bacterial probiotics, such as a lactic acid bacterium, e.g., L. lactis, will also be apparent to the skilled person.

For example, the promoter is a constitutive promoter. Exemplary constitutive promoters include:
The promoter from the rpoD gene of L. lactis (De Vos and Simons, Gene cloning and expression systems in Lactococci. In Genetics and Biotechnology of Lactic Acid Bacteria. Edited by Gasson M J, de Vos W M. Oxford: Chapman and Hall; 1994: 52-105);
synthetic derivatives derived from the rpoD gene of L. lactis and other genes in L. lactis (Jensen and Hammer Appl Environ Microbiol 63: 82-87, 1998); or
one of P21, P23, P32, P44 or P59, e.g., as described in van der Vossen et al., Appl Environ Microbiol 53: 2452-2457, 1987.

In another example, the promoter is an inducible promoter. Exemplary inducible promoters include:
sugar inducible promoters, such as:
   the Streptococcus thermophilus lac promoter;
   the L. lactis lac operon (Wells et al., Mol Microbiol 8: 1155-162, 1995); and
   the Lactobacillus pentosus xylA promoter (Lokman et al., J. Bacteriol., 179: 5391-5397, 1997);
heat inducible promoters such as involved in the rro inducible system (Nauta et al., Nat Biotechnol., 15: 980-983, 1997; and
Nisin-inducible promoters.

In one example, the inducible promoter is a nisin-inducible promoter. Exemplary nisin-inducible promoters are the nisA and nisF promoters and functional fragments thereof. For example, a nisA promoter can comprise the sequence from positions −156 to +156 with respect to the nisA transcription site, including transcription site, −35 and −10 sequences, NisR binding site and the RBS. Exemplary nisin-inducible promoters are described in Mierau and Kleerebezem Appl. Microbiol. Biotechnol., 68: 705-717, 2005 and/or Zhou et al., Biotechnol. Advances, 24: 25-295, 2006.

### Vectors

As will be apparent to the skilled person from the foregoing discussion, a nucleic acid encoding a polypeptide (e.g., a PAL or another polypeptide expressed in a probiotic) is operably linked to a promoter that can be within an expression vector. Suitable promoters will be apparent to the skilled person and/or described herein.

Expression vectors for expression in yeast cells are preferred and include, but are not limited to, the pACT vector (Clontech), the pDBleu-X vector, the pPIC vector suite (Invitrogen), the pGAPZ vector suite (Invitrogen), the pHYB vector (Invitrogen), the pYD1 vector (Invitrogen), and the pNMT1, pNMT41, pNMT81 TOPO vectors (Invitrogen), the pPC86-Y vector (Invitrogen), the pRH series of vectors (Invitrogen), pYESTrp series of vectors (Invitrogen). A number of other vector systems for expressing a polypeptide in yeast cells are well-known in the art and are described for example, in Giga-Hama and Kumagai (In: Foreign Gene Expression in Fission Yeast: Schizosaccharomyces Pombe, Springer Verlag, ISBN 3540632700, 1997) and Guthrie and Fink (In: Guide to Yeast Genetics and Molecular and Cell Biology Academic Press, ISBN 0121822540, 2002).

For expression in a bacterial probiotic, such as a lactic acid bacterium, plasmids have been constructed for intracellular production or secretion of the gene product. For the Nisin-induced expression system, pNZ8048 is one of the most commonly used plasmid for expression. Variants of this plasmid have been constructed, such as pNZ8148. In pNZ8148 a small 60-bp remnant DNA-fragment of Bacillus subtilis, the initial cloning host of the pSH series of plasmids, was removed, making the plasmid conform to self-cloning guidelines. Other plasmids include pNZ8110, which includes the secretion signal sequence of the major secreted protein Usp45 of L. lactis.

Additional vectors for expression in lactic acid bacteria are known in the art and described, for example, in Yeng et al., African J Biotechnol., 8: 5621-5626, 2009; Shareck et al., Crit. Rev. Biotechnol., 24: 155-208, 2004; and De Vos and Simmons 1994). Gene cloning and expression systems in lactococci. In Gasson and De Vos., Genet. Biotechnol. Lactic Acid Bacteria, pp. 52-105. Glasgow, UK: Chapman and Hall.

### Encapsulation

In a further example, the probiotic is encapsulated, e.g., microencapsulated.

Encapsulation of the probiotic can enhance survival in the gastric and/or gastrointestinal tract of a subject.

Reagents and methods of encapsulation are known in the art and/or described herein.

Exemplary reagents for encapsulation include alginate. Alginate is one of the most commonly used reagents for encapsulation of probiotics is alginate a linear polysaccharide consisting of 1→4 linked β-(D)-glucuronic (G) and α-(L)-mannuronic (M) acids generally derived from brown algae or bacterial sources. It is commercially available in a wide range of molecular weights from tens to hundreds of kilodaltons and is well suited to bacterial encapsulation due to its mild gelling conditions, GRAS (generally recognised as safe) status, and substantial lack of toxicity.

Alginate gels upon contact with divalent metals (e.g. calcium, cadmium or zinc). This ability has been exploited to form microcapsules using an extrusion process. This process involves the dropping of a concentrated alginate solution, most commonly through a needle, into calcium chloride solution, externally gelling the polymer into a microcapsule. The size of the microcapsules formed using external gelation is governed by the size of droplets formed during the extrusion process, with particles from as little as tens of microns being produced by spray technology, up to millimeter size when needle extrusion is used.

Another approach which is commonly used is the emulsion method. In this process microcapsules are formed by the formation of a water-in-oil emulsion, usually stabilised by surfactants, such as Tween 80, with the alginate being dissolved in the water phase. The alginate is usually then gelled by external gelation, i.e. the addition of calcium chloride solution to the emulsion. Alternatively, microcapsules may be formed by internal gelation, in which the alginate in solution contains calcium carbonate. An organic acid is added to this emulsion, and as it penetrates into the discrete water phase it reacts with the calcium carbonate forming calcium ions and carbonic acid, resulting in the gelation of the alginate.

The coating, or incorporation, of other materials into the alginate microcapsules can also be useful for probiotic microencapsulation research. Along with the protection that such coatings can offer to the microorganisms, other beneficial properties may also be imparted, such as giving greater control over release. A common coating material is the polysaccharide chitosan. Chitosan is a natural, linear cationic polysaccharide containing both glucosamine and N-acetyl glucosamine residues. Chitosan is the (usually partially) N-deacetylated form of chitin, a natural mucopolysaccharide derived from some natural supporting structures, such as the exoskeletons of crustaceans.

Other casting materials that can be combined with the alginate (or other encapsulating reagent) include whey protein, palm oil, xanthan gum, cellulose acetate phthalate or, starch.

Other polysaccharides that have been used to encapsulate probiotics include xanthan gum, gum acacia, guar gum, locust bean gum, and carrageenan.

### Testing for Activity

Methods for testing a probiotic for activity, e.g., PAL activity will be apparent to the skilled artisan based on the disclosure herein.

### PAL Activity

In one example, a probiotic is assessed for PAL activity.

As exemplified herein, one method for assessing PAL activity is to contact a probiotic with a composition comprising phenylalanine and assess levels of transcinnamate. Such a process may involve initially extracting transcinnamate and then determining levels of transcinnamate, e.g., by spectrophotometry. The level of activity may be determined by comparison to a known amount of recombinant PAL.

A kit for determining PAL activity is also commercially available from Sigma Aldrich.

In a further example, phenylalanine is administered to a subject (e.g., an animal) either with or without the probiotic. The level of phenylalanine is then determined in the blood of the subject. A reduced level of phenylalanine in the blood of a subject in the presence of the probiotic compared to in the absence of the probiotic indicates that the probiotic produces a polypeptide having PAL activity. Such a method is exemplified herein and/or described in Wibrand Clinica Chimica Act 347: 89-96, 2004.

### Viability

In another example a probiotic is assessed for viability, e.g., following exposure to conditions representative of conditions to which it will be exposed in vivo. For example, the probiotic is exposed to one or more or all of the following conditions:
pH5.0 in the presence of 0.01% lysozyme and 0.3% pepsin (exposure can be for between 5 minutes and 2 hours, such as about 20 minutes)—mimicking the upper stomach of a human;
pH4.1 (exposure can be for between 5 minutes and 2 hours, such as about 20 minutes)—mimicking the middle stomach of a human;
pH3.0 (exposure can be for between 5 minutes and 2 hours, such as about 20 minutes)—mimicking the middle stomach of a human;
pH2.1 (exposure can be for between 5 minutes and 2 hours, such as about 20 minutes)—mimicking the lower stomach of a human;
pH6.5 (exposure can be for between 5 minutes and 9 hours, such as about 20 minutes)—mimicking the proximal duodenum of a human; and/or
pH8.0 in the presence of 0.45% bile salts and 0.1% pancreatin (exposure can be for between 5 minutes and 9 hours, such as about 120 minutes)—mimicking the duodenum of a human.

In one example, the probiotic is exposed to all of the foregoing conditions.

Following the exposure, viability of the probiotic is assessed.

In one example, viability is assessed by culturing the probiotic under standard conditions and determining whether or not the probiotic is able to grow, with the identity of the genetically modified probiotic being confirmed by a specific PCR-based assay.

In another example, viability is assessed by culturing the probiotic in M17 media containing oxgal and determining whether or not the probiotic is able to grow. For example, the oxgal is present in the amount of at least 0% or 0.4% or 0.6% or 0.8% or 1%.

In another example, viability is determined by assessing whether or not there are any live cells in a sample of the probiotic, e.g., using a commercially available kit, such as the LIVE/DEAD® BacLight™ Bacterial Viability and Counting kit (Molecular Probes).

### In Vivo Animal Models

In a further example, a probiotic of the disclosure is tested for therapeutic or prophylactic effects in an animal model of PKU.

Animal models are known in the art and/or exemplified herein. Exemplary models include:
ethylnitrosourea (ENU) mouse models of PKU designated PAH^{enu1}, PAH^{enu2} and PAH^{enu3} as described in McDonald et al., Proc. Natl. Acad. Sci USA, 87: 1965-167, 1990 and Shedlovsky et al., Genetics, 134: 1205-1210, 1993 and crosses thereof; and
rats fed analogs of phenylalanine that inhibit PAH, e.g., as described in Lane et al., Biochim. Biophys. Acta. 627: 144-156, 1980.

Phenylalanine can be administered to such an animal model. Simultaneously or following phenylalanine administration, a probiotic of the disclosure is also administered. A reduction of phenylalanine levels in the animal model in the presence of the probiotic compared to in the absence of the probiotic indicates that the probiotic is useful for treating or preventing PKU.

### Compositions

The disclosure also provides a composition comprising a probiotic of the disclosure and a pharmaceutical composition comprising such a probiotic and a pharmaceutically acceptable carrier. Such compositions are useful as dietary adjuncts or pharmaceutical preparations which can be administered to PKU patients to treat PKU.

In one example, the composition comprises an inert carrier and/or a carrier to facilitate the probiotics being delivered to the gastro-intestinal tract (e.g., the small intestine) in a viable and metabolically-active condition. In one example, the cells are also delivered in a condition capable of colonising and/or metabolising and/or proliferating in the gastrointestinal tract.

In one example, the composition is a foodstuff. In this regard, the term “foodstuff” as used herein includes liquids (e.g., drinks), semi-solids (e.g., gels, jellies, yoghurt, etc) and solids. For example, the composition may be a flavoured jelly, e.g., to enhance the desirability of consumption, e.g., strawberry flavoured jelly. Exemplary foodstuffs include dairy products, such as fermented milk products, unfermented mild products, yoghurt, frozen yoghurt, cheese, fermented cream, milk-based desserts milk powder, milk concentrate or cheese spread. Other products are also contemplated, such as soy-based products, oat-based products, infant formula and toddler formula.

The composition can also be presented in the form of a capsule, tablet, syrup, etc. For example, the composition can be a pharmaceutical composition. Such a composition can comprise a pharmaceutically acceptable carrier, e.g., to facilitate the storage, administration, and/or the biological activity of the probiotic (see, e.g., Remington's Pharmaceutical Sciences, 16th Ed., Mac Publishing Company (1980). Suitable carriers for the present disclosure include those conventionally used, e.g., water, saline, aqueous dextrose, lactose, a buffered solution, starch, cellulose, glucose, lactose, sucrose, gelatin, malt, rice, flour, and the like.

Exemplary carriers do not adversely affect the viability of a probiotic.

In one example, the carrier provides a buffering activity to maintain the probiotic at a suitable pH to thereby exert a biological activity

The composition can comprise additional components, such as vitamins, such as vitamins of the B group, one or more minerals, such as calcium or magnesium, one or more carbohydrates, such as lactose, maltodextrin, inulin, dextrose, mannitol, maltose, dextrin, sorbitol, fructose, and a mixture thereof.

### Methods of Treatment

In one example, the present disclosure additionally provides a method for reducing levels of phenylalanine in a subject having PKU or preventing an increase in levels of phenylalanine in a subject having PKU after consuming a phenylalanine-containing foodstuff, the method comprising administering to the subject a probiotic of the disclosure or an encapsulated form thereof or a composition of the disclosure.

The present disclosure also provides a method for treating or preventing a symptom of PKU in a subject, the method comprising administering to the subject a probiotic of the disclosure or an encapsulated form thereof or a composition of the disclosure.

In one example, the administration is by ingestion, e.g., is oral.

The method of the disclosure clearly contemplates multiple administrations of the probiotic or encapsulated form thereof. For example, the probiotic or encapsulated form thereof may be administered on a daily basis or more or less often, depending on the survival of the probiotic in the subject.

In one example, the subject is human. For example, the subject is an infant human, or a human child or a human adult.

In one example, the probiotic, encapsulated form or composition is administered with food or within three hours or two hours or one hour of consuming food. Consuming the probiotic with food or soon thereafter is likely to increase the survival of the probiotic by increasing the pH of the acidic components of the gastric or gastrointestinal tract.

In one example, the probiotic, encapsulated form or composition is administered in an effective amount or a therapeutically effective amount or a prophylactically effective amount.

In one example, the method comprises administering the probiotic, encapsulated form thereof or composition in an effective amount of at least about 10⁴ to about 10¹⁰ cfu per dose; or about 10⁵ to about 10⁹ cfu per dose; or about 10⁵ to about 10⁷ cfu per dose; or about 10⁹ cfu per dose.

By “effective amount” is meant an amount sufficient to reduce phenylalanine levels in the blood of a subject. The amount to be administered to a subject will depend on the level of phenylalanine in their blood and/or digestive system, whether or not they have recently consumed food, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present disclosure to a specific quantity, e.g., weight or amount of probiotic, rather the present disclosure encompasses any amount of the probiotic sufficient to achieve the stated result in a subject.

The term “therapeutically effective amount” is the quantity which, when administered to a subject in need of treatment, improves the prognosis and/or state of the subject and/or that reduces or inhibits one or more symptoms of PKU to a level that is below that observed and accepted as clinically diagnostic or clinically characteristic of PKU. A person skilled in the art will be able to determine appropriate dosages depending on these and other factors. Accordingly, this term is not to be construed to limit the present disclosure to a specific quantity, e.g., weight or amount of probiotic, rather the present disclosure encompasses any amount of the probiotic sufficient to achieve the stated result in a subject.

As used herein, the term “prophylactically effective amount” shall be taken to mean a sufficient quantity of a probiotic to prevent or inhibit brain damage caused by chronically elevated levels of phenylalanine in a subject's brain. This term is not to be construed to limit the present disclosure to a specific quantity, e.g., weight or amount of probiotic, rather the present disclosure encompasses any amount of the probiotic sufficient to achieve the stated result in a subject.

In each of the above examples, if the probiotic is provided as a foodstuff, the dosage may be altered by changing the amount of the foodstuff consumed. The probiotic can also be administered by adding it to food prior to consumption.

Alternatively, the probiotic can be administered by gavage or other method suitable for delivery to the gastrointestinal tract, e.g., the small intestine.

The present disclosure also provides a method of preparing a foodstuff (e.g., a protein-containing food) having a reduced content of phenylalanine. This method comprises contacting a foodstuff containing phenylalanine with the probiotic of the disclosure for a period of time that is sufficient to convert at least part of the phenylalanine content of the foodstuff into compounds that do not cause PKU. For example, at least about 10% of the phenylalanine content is converted, such as at least about 15%, 20%, 25%, 30%, 50% or 90%.

Any foodstuff for which there is a need to reduce the phenylalanine content, can be treated by the above method. Thus, the foodstuff subjected to treatment can be selected from a vegetable material, an animal material including a milk protein such as a casein, a globulin or a whey protein, and a microbially-derived foodstuff.

### Articles of Manufacture

The present disclosure also provides an article of manufacture comprising a probiotic of the disclosure or an encapsulated form thereof or a composition of the disclosure.

The article of manufacture can be a storage container, for example, for storing a foodstuff comprising the probiotic, encapsulated form or composition.

The article of manufacture can be a storage container for storing a pharmaceutical composition, e.g., under sterile conditions.

Optionally, the article of manufacture is packed with instructions for use in a method of the disclosure.

The present disclosure includes the following non-limiting examples.

### Example 1

### Materials and Methods

1.1 Biochemical detection methods
1.1.1 Blood collection

Blood samples were collected from tail veins of mice by performing a small cut along the tail of the mice with a blade then collecting the blood (approximately 50 μl) using an ammonium heparinised micro haematocrit capillary tube (Chase Scientific). The blood was then immediately applied onto newborn screening cards (Whatman 903™ paper) and placed horizontally overnight until dried.

### 1.1.2 Dried Blood Spot Sample Preparation

Dried blood spots (DBS) collected on newborn screening cards, were punched with a hole puncher, creating a 3 mm diameter spot (equivalent to 3 μl). Samples were placed in a 96-well plate and were eluted in 250 μl internal standards L-phenylalanine and L-tyrosine (both labelled at 13C6, Cambridge Isotope Laboratories; 10 μM each) by placing on shaker (DELFIA Plate Shake) for 1 hr. Two hundred and forty microlitres of the eluted standards, test samples or control samples were then transferred to a new 96-well plate and air-dried on a dry block heater (Thermoline Scientific) at 40° C. Samples were then reconstituted in 100 μl of the mobile phase (50% acetonitrile: 50% water: 0.1% formic acid) and placed on the tandem mass spectrophotometer (Waters 1525μ, Binary HPLC Pump). Samples were analysed using MassLynx analysis software (V4.1).

1.1.3 Transcinnamate Extraction from Growth Media

Transcinnamate is the end product of the catabolic reaction catalysed by PAL and can be measured by enzymatic assay using a spectrophotometric method (see below). However, direct detection of transcinnamate from the growth media is not possible due to culture media interference with the spectrophotometric reading of the enzymatic assay. To overcome this problem, we opted to extract the transcinnamate from the broth using the solvent ethyl acetate and then take spectrophotometric readings of the extracted transcinnamate.

Half ml samples and standards are added to 0.5 g NaCl. A further 0.5 ml milliQ water was added followed by 50 μl 6M HCl. Two ml ethyl acetate was then added and the tubes were placed on a shaker (Ratek) for 2 min. Samples were spun at 750 rpm for 5 min and the supernatant was transferred to a new tube. Absorbance of transcinnamate was then measured at 290 nm in a 1 ml glass cuvette (Hellma 104-OS; 10 mm) using a spectrophotometer (Lightwave, Diode-array s2000 UV/VIS). The standards from the extraction method were standardised against standards in Tris-HCl buffer at pH 8.5.

### 1.2 Bacterial Strains and Growth Conditions

The bacterial strains and plasmids used in this study are listed in Table 1. E. coli strains were grown in Luria-Bertani (LB) medium at 37° C. with vigorous shaking. L. lactis strains were grown in M17 medium (Oxoid) containing 0.5% glucose (GM17) at 30° C. without shaking. E. coli transformation was performed by following the supplier's instructions. Electroporation of L. lactis was performed essentially as described elsewhere (Holo et al, Allp Envirom Microbiol; 55(12):3119-3123, 1989; Holo, Methods Mol Biol; 47:195-199, 1995; McIntyre et al, Appl Environ Microbiol, 55(3):604-610, 1989), and transformants were plated on GM17 agar plates containing the required antibiotics. Plasmid constructions were maintained by the addition of 10 μg/ml of chloramphenicol for both E. coli and L. lactis strains. Erythromycin was used at a final concentration of 5 μg/ml for L. lactis.

TABLE 1
Strains and plasmids used in the present examples

| | | |
|---|---|---|
| Strain/ | | |
| Plasmid | Description | Source |

Strain

| | | |
|---|---|---|
| E. coli | F- Φ80lacZΔM15 Δ(lacZYA-argF) U169 | Invitrogen |
| DH5α | recA1 endA1 hsdR17 (rK−, mK+) phoA | |
| | supE44 λ- thi-1 gyrA96 relA1 | |
| E. coli | F- mcrA Δ(mrr-hsdRMS-mcrBC) | Invitrogen |
| TOP10 | φ80lacZΔM15 ΔlacX74 recA1 | |
| | araD139 Δ(ara-leu)7697 galU galK | |
| | rpsL (StrR) endA1 nupG | |
| L. lactis | MG1363 (nisRK genes into chromosome), | |
| NZ9000 | plasmid free | |

Plasmid

| | | |
|---|---|---|
| pNZ8148 | broad host range vector; CmR; nisA | NIZO |
| | promoter | |
| pCYT:Nuc | pWV01 derivative, Nuc gene expressed | |
| | under PnisA | |
| pCYT-PAL | pWV01 derivative, PAL gene expressed | |
| | under PnisA | |
| pMK | Cloning vector containing codon-optimised | GeneART |
| | PAL gene | |
| pCMK | pWV01 derivative, codon-optimised PAL | |
| | gene expressed under PnisA | |
| pMSP3535H3 | pJH24 derivative, nisRK, nisL | |
| pMCMK | Codon-optimised PAL gene expressed under | |
| | PnisA in pMSP3535H3 | |
| pSMC | Codon-optimised PAL gene expressed under | |
| | PnisA in pMSP3535H3 | |

### 1.3 DNA Isolation, Manipulation, and Synthesis

DNA manipulation was performed essentially as described (Sambrook et al, Molecular Cloning: A Laboratory Manual; 2001). Plasmid DNA from E. coli was isolated with alkaline lysis essentially as described (Sambrook et al, Molecular Cloning: A Laboratory Manual; 2001) or using a QIAprep Spin Miniprep Kit (QIAGEN). An extra step was carried out for L. lactis plasmid DNA isolation (Le Loir et al, Journal of bacteriology, 180(7):1895-1903, 1998). L. lactis cells were suspended in TES buffer (25% sucrose, 1 mM EDTA, 50 mM Tris-HCl; pH 8) containing lysozyme (10 mg/ml) and incubated for 30 min at 37° C. to prepare protoplasts, followed by the standard alkaline lysis. The codon-optimised PAL gene was synthesized by GeneArt (Germany).

### 1.4 Plasmid Construction

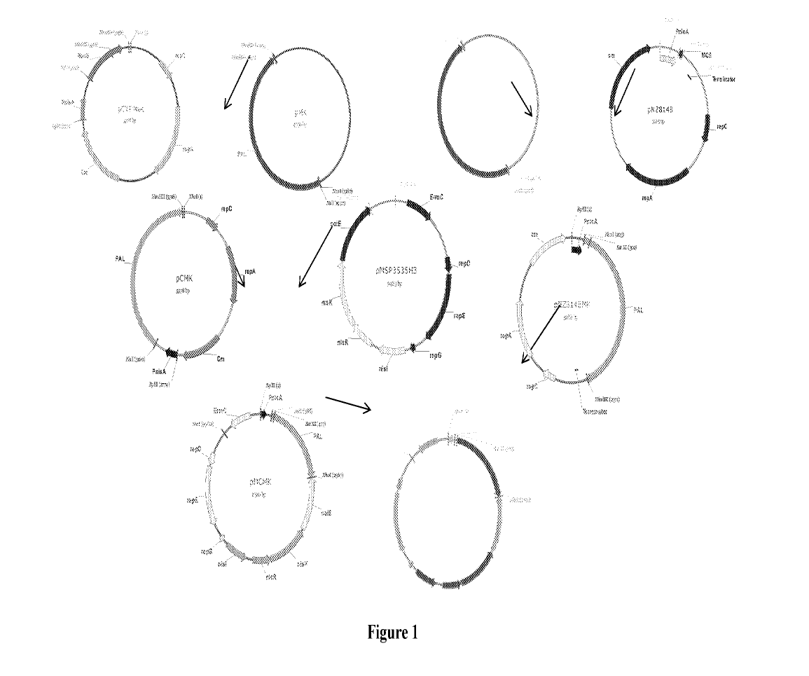

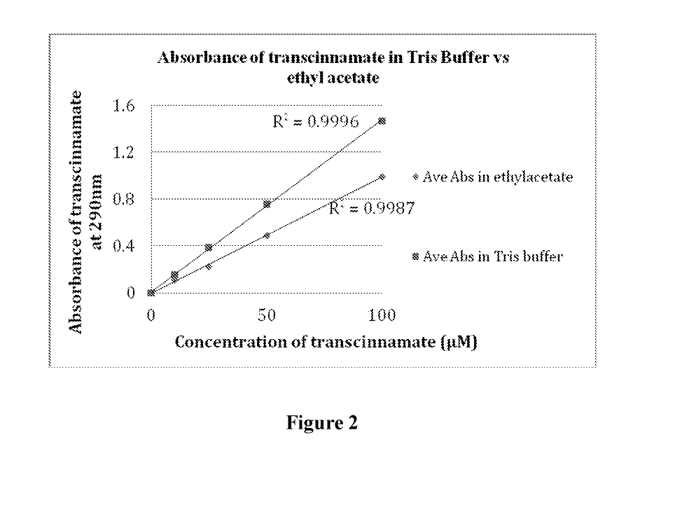

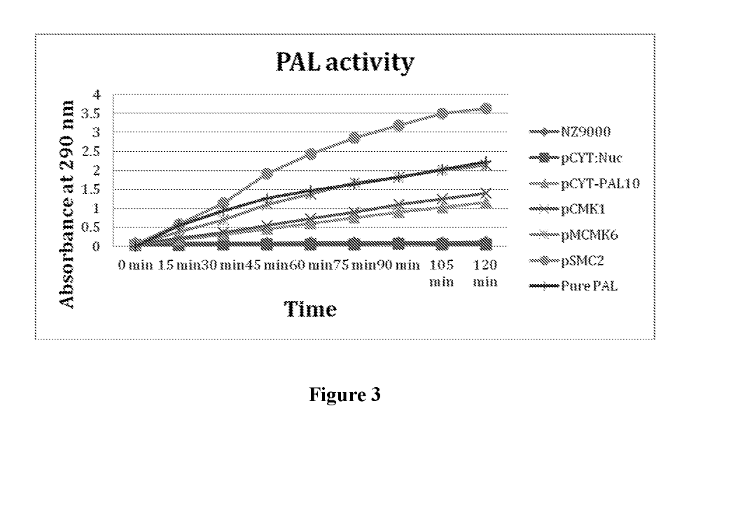

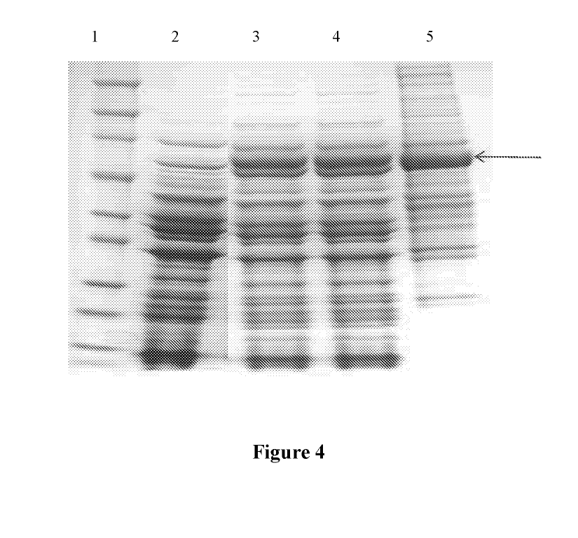

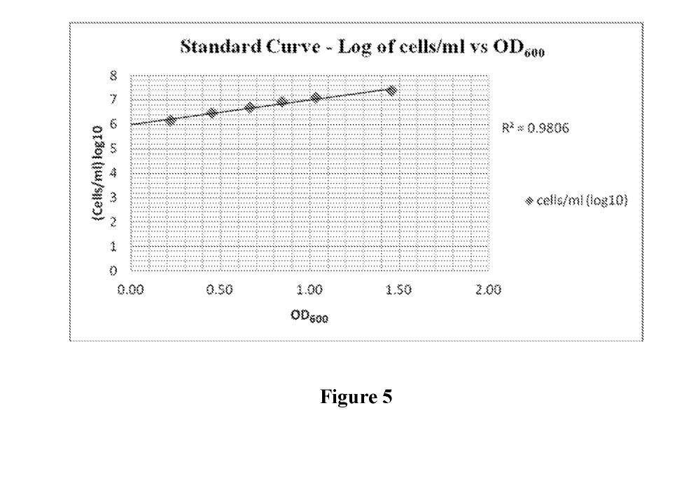

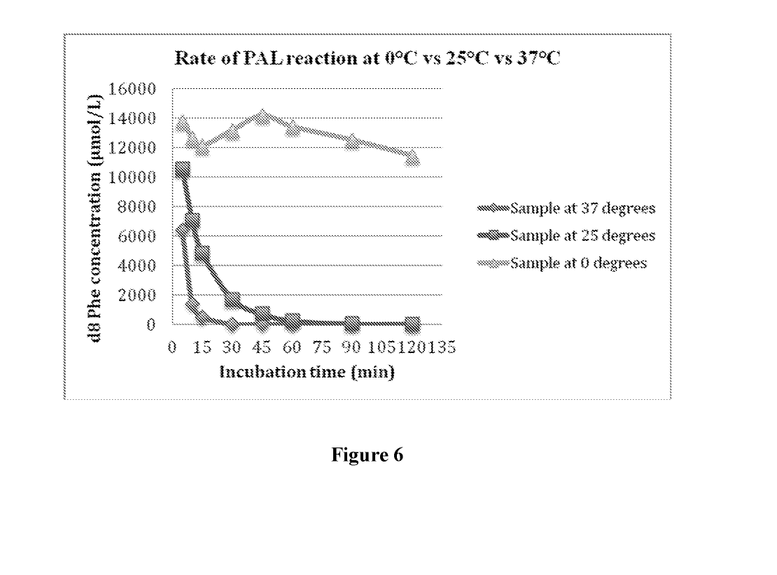

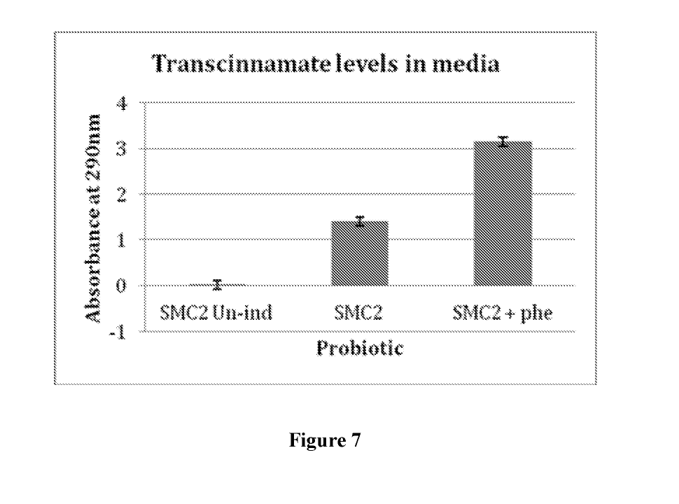

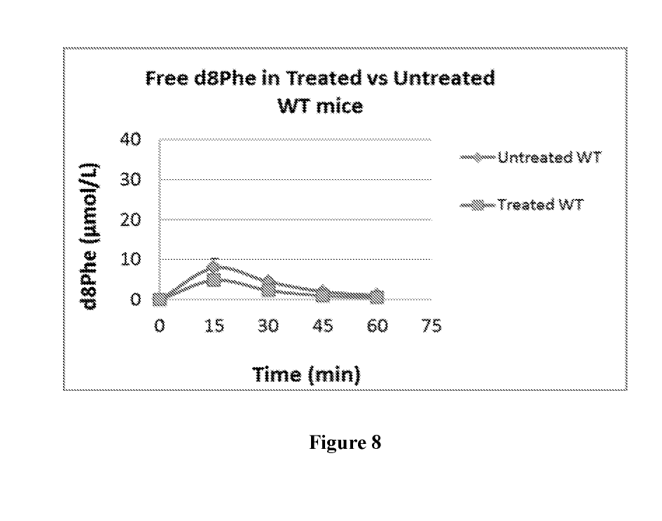

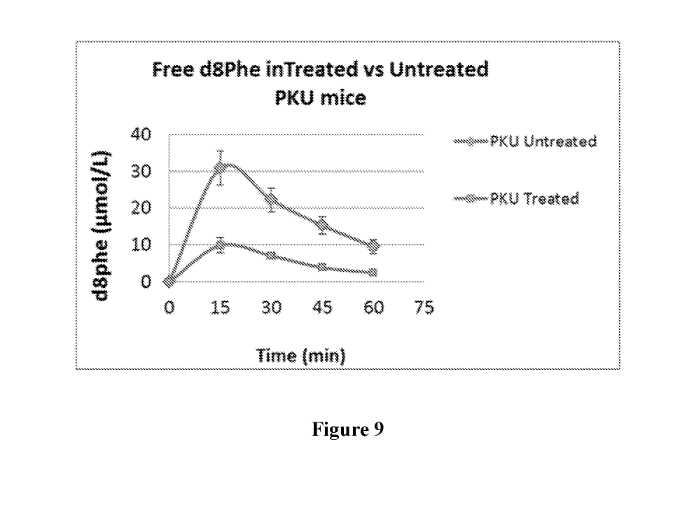

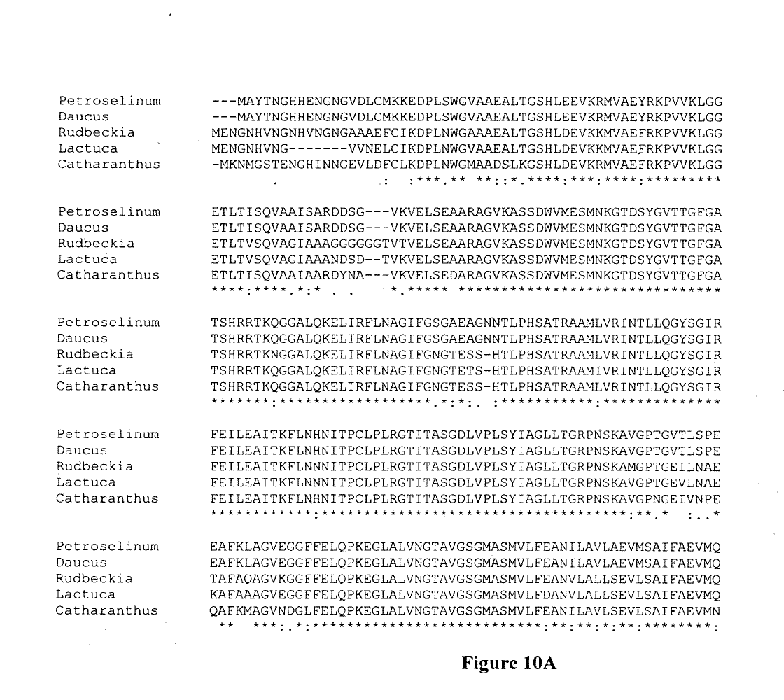

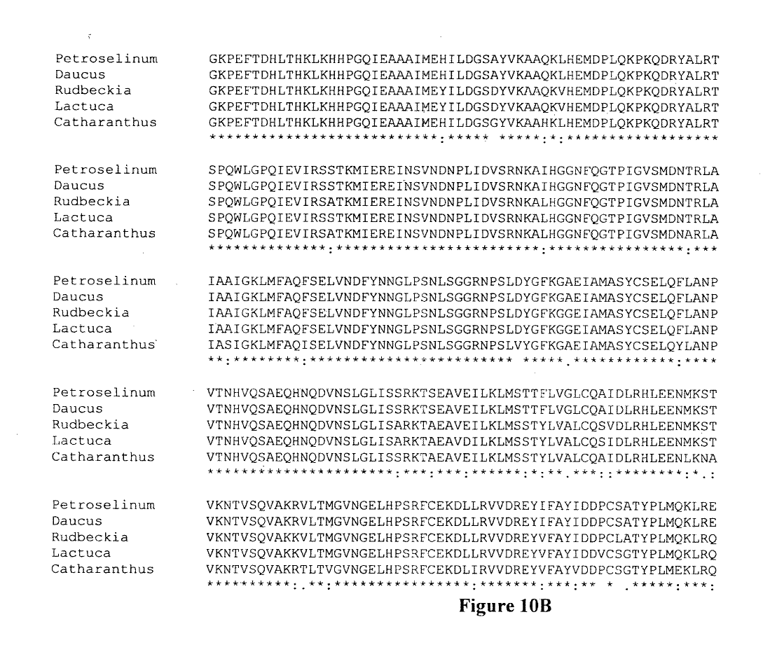

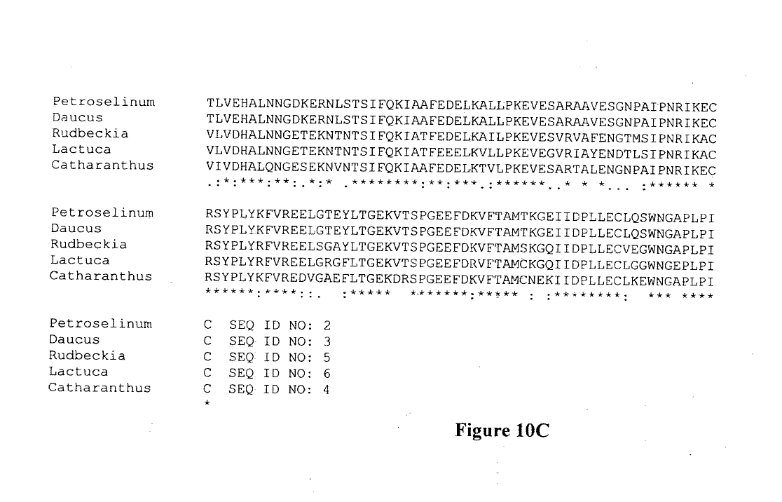

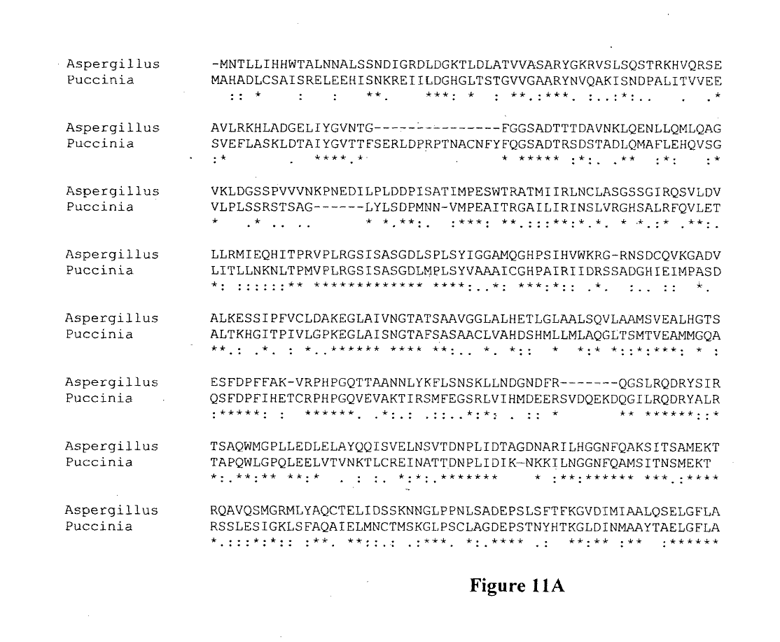

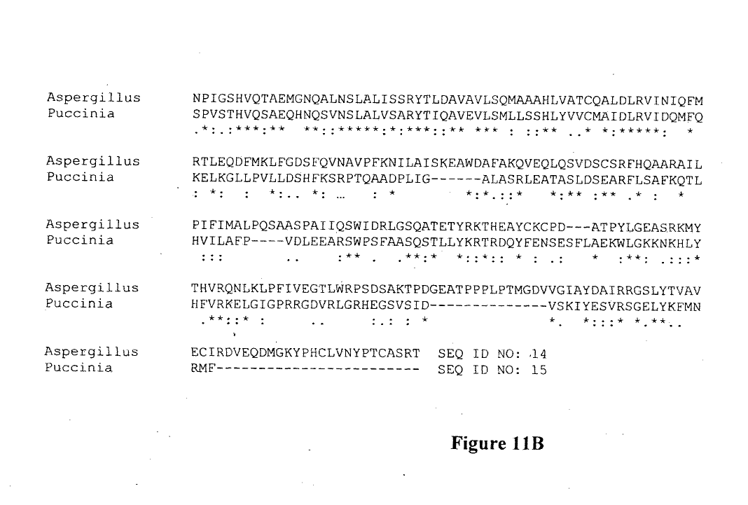

A summary of the sequence of steps used for the construction of expression vectors is shown in FIG. 1.

The plasmid pT7-7, which contains the Petroselinum crispum PAL gene, was a gift from Dr. Schulz (Baedeker et al, FEBS Lett; 457(1):57-60, 1990.). The plasmid pCYT:Nuc, which expresses Staphylococcal nuclease under the control of the nisin inducible promoter PₙᵢₛA, was a gift from Dr. Bermudez-Humaran (Bermudez-Humaran et al, FEMS Microbiol Lett, 224(2):307-313, 2003). The NsiI/XhoI fragment containing the nuclease coding sequence in pCYT:Nuc was replaced by the PAL coding sequence as an NsiI/XhoI fragment from pT7-7 and transformed into the Lactococcus strain NZ9000 (NIZO, the Netherland), resulted the vector pCYT-PAL. To obtain the higher protein expression, a new PAL coding sequence was synthesised with the preferred codon usage for Lactococcus (by GENEART, Germany). The PAL coding sequence in the vector pMK (from GENEART) was excised as an NsiI/HindIII fragment and inserted into the NsiI/HindIII digested pCYT:Nuc, resulting in the vector pCMK (FIG. 1).

The plasmid pMSP3535H3 was obtained from Dr. Mills (Oddone et al., Plasmid. [Research Support, Non-U.S. Gov′t]. May; 61(3):151-158, 2009). The nisA promoter and the codon optimised PAL coding sequence from pCMK were excised as a BglII/XhoI fragment and cloned into the BglII/XhoI digested pMSP3535H3, resulting in the vector pMCMK (FIG. 1).

The codon-optimised PAL sequence was also cloned into plasmid pNZ8148 (NIZO, the Netherlands) as an NcoI/HindIII fragment to generate the pNZ8148MK vector. A BglII/BstXI fragment containing nisA promoter and a part of PAL coding sequence was then excised from p8148MK and cloned into the pMCMK that had been already cut by BglII and BstXI to generate the pSMC2 vector (FIG. 1).

Wherever possible, the cloning work was carried out using Lactococcus lactis NZ9000 as the host. For some difficult cloning work, E. coli strain DH5α or Top10 cell (Invitrogen) were used as the hosts and the generated plasmids were further transformed to Lactococcus lactis NZ9000.

### 1.5 Nisin Induction and Cell Lysate Preparation

Nisin induction was performed as described (de Ruyter et al, Appl Environ Microbiol; 62(10):3662-3667, 1996). An overnight culture of L. lactis strains was inoculated into a fresh M17 medium in a 1/25 ratio and incubated at 30° C. until an OD₆₀₀ of 0.4-0.6 was attained. Nisin induction was carried out by adding 1-10 ng/ml of nisin (Sigma) and the incubation was continued for another 4 hours. Cells (from 1 ml culture) were collected by centrifugation with a desktop centrifuge at top speed for 1 minute, washed twice with Tris buffer (150 mM Tris, pH 8.5) and resuspended in 100 μl of Tris buffer. Cell lysates were prepared using a Branson sonifier 250 (Branson Ultrasonic SA, Switzerland) for 10 bursts at an output setting of 4 and 40% duty cycle, followed by a 10 minute centrifugation in a desktop centrifuge at top speed at 4° C.

### 1.6 PAL Activity Assay

PAL activity was studied by monitoring transcinnamic acid formation in a spectrophotometer (Hodgins, J Biol Chem; 246(9):2977-2985, 1975.). Briefly, 20 μl of a cell lysate was added into 1 ml of 10 mmol phenylalanine in Tris buffer (150 mM Tris, pH 8.5), mixed by inversion and incubated at 37° C. A 100 μl aliquot was taken out of the reaction mix to measure the absorbance at 290 nm at 15-minute intervals using a Beckman DU 650 spectrophotometer (Beckman Coulter Australia Pty Ltd, Australia). Two μg of purified phenylalanine ammonia-lyase from a yeast, Rhodotorula glutinis (product of Sigma), used as the positive control, was added into the same reaction volume.

### 1.7 Probiotic Cell Count and Viability

To accurately count cells and examine their viability, the LIVE/DEAD® BacLight™ Bacterial Viability and Counting kit (Molecular Probes; L34856) was used, following the manufacturer's instructions. This kit gives the ability to distinguish and quantitate live and dead bacteria with the aid of a flow cytometer (BD FACSCanto™)

### 1.8 In Vitro Study of PAL Activity in Live Probiotic Bacteria at Varying Temperatures

To investigate the rate of the PAL reaction at different temperatures the decrease in phenylalanine labelled with a stable isotope, deuterium 8 (d8phe) was measured using tandem mass spectrometry (TMS).

Five hundred ml of probiotic culture was harvested (containing a total of 6×10⁹ cells) by centrifugation at 5000 g for 5 min. Three ml of 1 mg/ml of labelled phenylalanine (d8phe) was added to 0.3 g skim milk powder. One and a half ml of the ice-cold labelled isotope/skim milk suspension was added to make up a total volume of 3 ml of probiotic and labelled isotope mixture. All of the foregoing steps were performed on ice.

Three separate aliquots of 1 ml were taken, where one aliquot was kept in ice (0° C.), one at room temperature, and the third aliquot at 37° C. Samples were incubated for a total of 2 hr, with 50 μl aliquots being taken at 5 min intervals for the first 15 min then every 15 min thereafter. Reactions at each time point were stopped immediately with 100 μl of ice-cold ethanol and spun at 14,000 rpm for 10 min at 4° C. Supernatants were transferred to 96-well plates and air dried. Two hundred and forty microliters of internal standard (2 μM L-phenylalanine 13C6 in methanol) was added to each well and then air-dried on dry block heater Thermoline Scientific) at 40° C. Samples were reconstituted in 100 μl of the mobile phase (50% acetonitrile: 50% water: 0.1% formic acid), then run and analysed on the TMS.

### 1.9 Simulated Investigation of the Survival of the Probiotic in Different Compartments of the Gastric and Gastrointestinal (G/GI) Tract

To examine the ability of the probiotic to survive in different compartments of the G/GI tract, a modified version of a previously published method (De Palencia et al, Eur Food Res Technol.; 227:1475-1484, 2008) was used. For the modified version, cells were kept in M17 media and were not reconstituted in milk. The probiotic was exposed to milieus representing different compartments of the G/GI tract. This method mimics the in vivo situation where there is a sequential decrease in pH. Samples were incubated for 20 min at conditions simulating exposure of the probiotic to the contents of the upper stomach to the proximal duodenum, and for a further 2 hr at conditions mimicking those in the distal duodenum. The total incubation time at which the probiotic was exposed to the different simulated compartments of the G/GI tract was three hours and twenty minutes. To examine culturability of the probiotic after being subjected to each stage of the G/GI clearance, 100 μl samples were withdrawn at each pH point, inoculated in selective media (M17 containing erythromycin) and cultured overnight.

1.10 Investigation of Whether the PAL Expressed by the L. Lactis is Able to Catabolise Phenylalanine Contained within Intact Polypeptides

L. Lactis was cultured in M17 media (which contains protein broth normally devoid of free L-phenylalanine), and the capacity of the probiotic to metabolise L-phenylalanine within intact polypeptides in the broth was assayed by measuring the generation of transcinnamate. The positive control was the same broth “spiked” with free L-phenylalanine (Sigma). Following one hour incubation at 37° C., transcinnamate was extracted from the broth as described earlier for analysis.

### 1.11 Investigation of the Acute Effect of the Genetically-Modified Probiotic In Vivo Using Our PKU Mouse Model.

### 1.11.1 Labelled Isotope Preparation and Administration

A load of the isotopically labelled phenylalanine (d8phe) was used in PKU and wild type (WT) mice. We also used tyrosine labelled with deuterium-4 (d4tyr) stable isotope as an internal standard to compare and correct for any absorption variability between phenylalanine and tyrosine. The labelled isotope mixture was prepared using 1 mg/ml of each d8phe and d4tyr (Cambridge Isotopes) in water and acidified with 6M HCl until completely dissolved (final pH was 2). Skim milk powder (0.5 g) was added to 5 ml of the labelled isotope mixture and was used as the vehicle to deliver the probiotic. Control untreated groups (5 WT and 5 PKU mice) received 0.5 ml of this mixture without the probiotic via orogastric gavage (a 22 ball point needle was used to perform the gavage).

### 1.11.2 Probiotic Preparation

For in vivo experiments, probiotic was prepared on a larger scale to obtain sufficient culture to deliver a dose of 10⁹ cells per mouse. For this purpose, L. lactis was grown overnight in 100 ml of M17 media containing 0.5% glucose and 100 μl erythromycin (5 mg/ml) at 30° C. The following day, the culture was added to fresh M17 media (1800 ml M17 and 100 ml 0.5% glucose) and placed on a shaker (Bioline) at 100 rpm at 30° C., and grown until the OD₆₀₀ reached approximately 0.4. The culture was then induced with 200 μl (10 mg/ml) nisin A (Nisaplin, Sigma), and incubated on the shaker for a further 4 hr at 30° C. The probiotic was checked for enzyme activity prior to use for in vivo experiments.

For each treated group, the induced probiotic was prepared by harvesting 500 ml of the induced culture (containing a total of 6×10⁹ cells) by centrifugation at 5,000 g for 5 min. Three ml of 1 mg/ml of each d8phe and d4tyr was added to 0.3 g skim milk powder. One and a half ml ice-cold labelled isotopes/skim milk solution was added to make up to a total volume of 3 ml probiotic and labelled isotope. This was prepared and kept on ice until being administered to the mice. The treated groups (5 WT and 5 PKU mice) received 0.5 ml of the labelled isotope mixture containing 10⁹ cells via orogastric gavage.

### Example 2

### Results

### 2.1 Transcinnamate Quantitation

Transcinnamate extracted from broth was estimated to be about 70% of the total transcinnamate present in the broth. The extraction efficiency of transcinnamate comparing ethyl acetate and Tris-HCl buffer (pH 8.5) is shown in FIG. 2.

### 2.2 Construction and Validation of PAL Expression Constructs

The Petroselinum crispum PAL coding sequence was initially cloned into the plasmid pNZ8148 using E. coli Top10 cells as the host. However, restriction analysis and sequencing revealed that a sequence re-arrangement occurred in this construct and made it unusable. Several other E. coli strains (E. coli DH5α, JC8111, XL-10 and Stb12) and Lactococcus lactis NZ9000 were tested as the hosts for this construct, but the problem persisted. Consequently, the PAL coding sequence was cloned into the plasmid pCYT:Nuc using Lactococcus lactis NZ9000 as the host to generate pCYT-PAL, in which PAL was also under the control of the NisA promoter. The cell lysates prepared from the nisin induced pCYT-PAL transformants showed a reasonable PAL activity level. To increase the PAL expression, a new PAL coding sequence was synthesized with codon usage modified according to Lactococcus preferences. This codon optimisation process did not alter amino acid sequence of the PAL protein. This PAL coding sequence was cloned into pCYT to generate pCMK. A slightly increase on PAL activity was observed for cell lysates using pCMK transformants compared to pCYT-PAL transformants.

The plasmid pMSP3535 was then developed by incorporation of the genes NisR and NisK into the original nisin-inducible expression vector. In a subsequent derivative, pMSP3535H3, the Nisi gene was further incorporated into the plasmid backbone to achieve higher protein expression because inclusion of this gene enabled the use of a higher nisin dose for induction (Oddone et al, supra). The fragment containing the PnisA promoter and the codon-optimised PAL coding sequence in pCMK was cloned into pMSP3535H3 to generate the pMCMK plasmid. Different nisin concentrations were tested in the induction of Lactococcus lactis NZ9000 harbouring pMCMK. It was found that this strain could tolerate a higher nisin concentration (10 μg/ml compared to 1 μg/ml) and had a higher PAL activity (about 2 fold) compared to the pCMK transformants.

A short fragment of nisA coding sequence between promoter PnisA and Nuc coding sequence in the plasmid pCYT:Nuc. It was incorporated into pCMK and pMCMK as part of the cloning procedures. It is not clear that what effect of the resulting fusion NisA-PAL protein would have on PAL activity. To eliminate this fragment, the codon-optimised PAL sequence from pMK was cloned into plasmid pNZ8148 in which the PAL is directly downstream of the promoter PnisA, to generate the pNZ8148MK vector. A short fragment containing the promoter PnisA and a part of PAL sequence was used to replace the same fragment containing the extra nisA sequence, to generate the pSMC2 vector. The PAL activity assay showed a further doubling of PAL activity with the pSMC2 transformants.

The PAL activity from each construct was compared. The pSMC2 transformants showed the highest PAL activity (FIG. 3; Table 2). The significant increase in PAL expression can also been seen on a Coomassie stain protein gel (FIG. 4).

TABLE 2
PAL enzyme activities of the various PAL expression constructs

| | Activity | |
|---|---|---|
| Expression Vector | (μmol/min/mg protein) | % of Pure PAL |
| NZ9000 | 0.000850 | 0.043 |
| pCYT | 0.00118 | 0.060 |
| pCYT-PAL10 | 0.0567 | 2.86 |
| pCMK1 | 0.0692 | 3.50 |
| pMCMK6 | 0.136 | 6.87 |
| SMC2 | 0.482 | 24.3 |
| Pure PAL | 1.98 | 100 |

L. lactis NZ9000 and pCYT were used as the negative controls. pCYT-PAL10, pCMK1, pMCMK6 and pSMC2 are the expression vectors constructed at different stages. The Pure PAL is the purified phenylalanine ammonia-lyase from a yeast Rhodotorula glutinis (Sigma).

### 2.3 Validation of Probiotic Cell Count and Viability Assay

A standard curve was established whereby different cell densities were prepared and analysed using spectrophotometric analysis at OD₆₀₀ as well as a flow cytometer (BD FACSCanto using FACSDiva software analysis V6.1.3). Using this approach an accurate quantitation of cells/ml of broth can be extrapolated (Table 3).

TABLE 3
Cell numbers of Lactococcus lactis and their corresponding OD₆₀₀

| average | Av. # Events of bacteria | cells/ml |
|---|---|---|
| OD₆₀₀ | (flow cytometer) | (log10) |
| 0.22 | 14200 | 6.15 |
| 0.45 | 29308 | 6.47 |
| 0.67 | 49868 | 6.70 |
| 0.85 | 82434 | 6.92 |
| 1.04 | 123049 | 7.09 |
| 1.46 | 242476 | 7.38 |

2.4 In Vitro PAL Activity within Live Probiotic Bacteria at Different Temperatures

FIG. 6 illustrates that the immediate drop in d8phe due to the PAL activity at 37° C. and 25° C. (room temperature). By 30 min, most of the d8phe is catabolised at 37° C. and by 60 minutes, a significant amount of the d8phe is catabolised at room temperature. The precise levels are shown in Table 4.

TABLE 4
PAL activity as indicated by concentration (μmol/L) of the d8phe over 2 hr at
different temperatures.

| Temp | Time (mm) | | | | | | | |
|---|---|---|---|---|---|---|---|---|
| (° C.) | 5 | 10 | 15 | 30 | 45 | 60 | 90 | 120 |
| 37 | 6392.7 | 1399.76 | 525.98 | 58.71 | 50.32 | 39.96 | 33.01 | 45.31 |
| 25 | 10561.8 | 7092.58 | 4856.38 | 1725.89 | 732.94 | 264.45 | 64.76 | 53.3 |
| 0 | 13781.1 | 12651.9 | 12126.8 | 13183.93 | 14197.3 | 13437.87 | 12524.39 | 11463.39 |

### 2.5 Survival of the Genetically-Modified Probiotic in Different Compartments of the (G/GI) Tract

Table 5 shows that the probiotic was able to survive after being subjected to environments that mimic different compartments of the G/GI tract. This is evident by the ability of the probiotic to be cultured when inoculated in selective media and allowed to grow overnight.

TABLE 5
Survival and culturability of the genetically-modified probiotic
after exposure to different compartments of the G/GI tract

| pH/Gastric/ | | | |
|---|---|---|---|
| Gastrointestinal | G/GI | Incubation time | |
| enzyme | compartment | at 37° C. (min) | Culturability |
| 0.01% Lysozyme + | Upper Stomach | 20 min | ✓ |
| 0.3% pepsin pH 5.0 | | | |
| pH 4.1 | Middle stomach | 20 min | ✓ |
| pH 3.0 | Middle stomach | 20 min | ✓ |
| pH 2.1 | Lower stomach | 20 min | ✓ |
| pH 6.5 | Proximal | 20 min | ✓ |
| | duodenum | | |
| 0.45% bile salts + | Duodenum | 120 min | ✓ |
| 0.1% Pancreatin | | | |
| (pH 8.0) | | | |

2.6 Capacity of PAL Expressed by the L. Lactis to Catabolise Phenylalanine Contained within Intact Polypeptides

FIG. 7 shows a significant production of transcinnamate in media containing no free L-phenylalanine, confirming that the probiotic can internalise protein in the broth, digest the protein to release L-phenylalanine and then catabolise it to transcinnamate.

### 2.7 In Vivo Acute Effect of the Genetically-Modified Probiotic Using a PKU Mouse Model

FIG. 8 shows the appearance and clearance of the labelled isotope (d8phe) in peripheral blood samples from treated and untreated wild type mice. The zero time point represents the blood concentration of the d8phe prior to the labelled isotope/milk (untreated) or isotope/milk/probiotic (treated) mix (sitting on ice) being administered, with the peak blood concentration being seen at approximately 15 min after administration by IG tube, and returning to zero by 60 min after administration.

The top curve (shown in diamonds) represents the untreated WT group, which reached a peak d8phe concentration of 8 μM at 15 min, while the highest concentration for the treated WT group (bottom curve, shown in squares) reached 5 μM at 15 min. These data indicate that the probiotic in the treated group was able to catabolise a portion of the labelled phe in the gut. However, WT mice have normal hepatic PAH activity, thus most of the d8phe is absorbed and consequently measured in the blood.

FIG. 9 shows the appearance and clearance of d8phe in the blood of PKU mice. The highest level of d8phe in the untreated group (top curve, diamonds) was 31 μM at 15 min while the highest concentration in the treated mice (bottom curve, squares) was 10 μM at 15 min. There was a significant difference (p<0.05; Mann-Whitney unpaired test) between the treated and untreated PKU mice. The difference was even greater when compared to the WT mice.

It is also worthwhile noting that the concentration of d8phe in untreated PKU mice is much higher than that seen in the untreated WT mice (although both groups received the same amount of the d8phe load). This is entirely to be expected. Following a protein load portal blood levels of amino acids are higher than in the peripheral circulation (Nässl et al, Am J Physiol—Gastrointestinal and Liver Physiology, 301(1):G128-G37, 2011), and given the PKU mice have hepatic deficiency of phenylalanine hydroxylase, we would expect peripheral blood d8phe levels to be higher in the PKU mice.

### Example 3

### Labelled Isotope Protein Load—Investigation of the Effect of the Probiotic on Labelled Phenylalanine Contained within Polypeptides In Vivo

Methods published by McIntosh and Dahlquist (McIntosh et al, Quart Rev Biophys. 1990; 23(1)) are used to characterise the effect of the probiotic on labelled phenylalanine contained within polypeptides in vivo. For specific amino acid labelling (i.e., d8phenylalanine and d4tyrosine) the protein is produced by expression in bacteria (e.g., E. coli), which will be grown on minimal medium supplemented with small amounts of ¹⁵NH₄Cl and ¹³C-labelled glucose, as well as labelled and unlabelled amino acids. The bacteria are then lysed and fed to mice as a labelled protein load. Probiotic is then fed to the mice and levels of labelled phenylalanine in circulation determined essentially as described above.

Alternatively, the protein (i.e., d8phenylalanine-labeled protein) is produced by expression in yeast (Saccharomyces cerevisiae), due to its high protein content. The yeast is grown in a nitrogen base media supplemented with native amino acids, except phenylalanine, which is replaced with deuterium 8 labelled phenylalanine. The yeast is then heat inactivated at 95° C. for 1 hour, concentrated by lyophilisation and fed to mice as a labelled protein load by orogastric gavage. Lyophilisation of the yeast protein allows administration of a higher dose of labelled protein in a reduced volume.

Probiotic is then fed to the mice and levels of labelled phenylalanine in circulation determined essentially as described above. Protein is extracted from the yeast by suspending 1 ml of yeast culture (containing approximately 10⁸ cells) in lysis buffer (0.1M NaOH, 0.05M EDTA, 2% SDS and 2% TCEP), and quantitated using a BCA protein assay kit.

### Example 4

### Improvement of Heterologous Protein Expression in the Probiotic

It is possible to increase the overall production of a recombinant protein in many systems by inducing secretion of the expressed protein out of the microorganism. The USP45 secretion signal is used to direct the secretion of the expressed PAL in Lactococcus. Some reports showed that a short synthetic peptide LEISSTCDA inserted between the secretion signal peptide and the expressed protein can increase protein production (Le Loir et al, Journal of bacteriology, 180(7):1895-903, 1998; Le Loir et al, Appl Environ Microbiol.; 67(9):4119-27, 2001). Another study has shown that a Lactobacillus brevis S-layer protein signal peptide directs stronger protein secretion than the USP45 signal peptide (Fernandez et al, Appl Environ Microbiol.; 75(3):869-71, 2009). The foregoing secretion signals are tested by introducing them into the PAL expression constructs described above.

To further increase the PAL expression, a nucleic acid encoding a chaperone protein is integrated into the constructs described above. For example, a nucleic acid encoding the Bacillus subtilis chaperone-like protein PrsA (Lindholm et al, Appl Microbiol Biotechnol.; 73(4):904-14, 2006) is integrated into the constructs. Total protein yield and secreted protein activity is confirmed using Western blotting techniques.

### Example 5

### Improvement of the Survival of the Probiotic in the GI Tract

The survival rate of Lactococcus in GI tract can be low (Vesa et al, Aliment Pharmacol Ther.; 14(6):823-828, 2000) (however, the probiotic of the present disclosure survives to a sufficient extent to provide a benefit), but has been found to be improved when taken with food, or by introducing a heterologous enzyme involving bile metabolism (Watson et al, BMC Microbiol. 2008; 8:176). By introducing a bilE gene (e.g., coding for a bile salt hydrolase from Listeria monocytogenes). A nucleic acid encoding a bile salt hydrolase is incorporated into an expression construct described herein. Viability of the genetically-modified, bilE enhanced probiotic is performed by subjecting the probiotics to different bile salt concentrations using M17 media containing oxgal. M17 media without oxgal is used as a control.

Alternatively, nucleic acid encoding the chaperone protein and bile salt hydrolase are inserted into the Lactococcus genome by using pGhost plasmid (Biswas et al, Journal of bacteriology. 1993; 175(11):3628-35) or the pNZ8148 plasmid. The in vitro PAL activity is tested using the spectrophotometric assay as described above.

### Example 7

### Genetically-Modified Lactobacillus

The survival rate of Lactobacillus in the GI tract, in particular the upper small intestine, has been found to be higher compared with Lactococcus strains (however, the Lactococcus strains of the present disclosure survives to a sufficient extent to provide a therapeutic benefit). Transforming Lactobacillus with the pSMC plasmid, where PAL is under the control of a nisin-inducible promoter, increases GI tract survival of the microorganism and intracellular PAL stability.

To further increase the survival rate of Lactococcus described previously or Lactobacillus in the GI tract, the probiotic is microencapsulated.

### Example 6

### Long-Term Effect of the Probiotic In Vivo

The genetically modified Lactobacillus or Lactococcus probiotic is able to survive after being subjected to environments that mimic different compartments of the mouse G/GI tract. By administering the probiotic in the form of strawberry flavoured jelly, with a range of organisms per dose, a plurality of times, a sustained positive effect on blood phenylalanine in the PKU mouse is provided.

1. A genetically-modified probiotic expressing recombinant phenylalanine ammonia lyase (PAL).

2. The probiotic of claim 1, which expresses PAL at a level such that when a composition comprising a plurality of the probiotics is administered to a subject with phenylalanine, the level of phenylalanine in the blood of the subject is significantly lower than the level observed in a subject to whom phenylalanine has been administered in the absence of the composition.

3. The probiotic of claim 1 or 2, which remains viable following exposure to a gastric and/or gastrointestinal tract of a subject or to an environment mimicking one or more conditions of a compartment of a gastric and/or gastrointestinal tract of a subject.

4. The probiotic of any one of claims 1 to 3, which is a bacterium.

5. The probiotic of claim 4, which is a lactic acid bacterium.

6. The probiotic of claim 4 or 5, which is of the genus Lactobacillus or Lactococcus.

7. The probiotic of any one of claims 4 to 6, which is Lactococcus lactis.

8. The probiotic of claim 1, wherein the PAL is from a plant.

9. The probiotic of claim 8, wherein the PAL is from Petroselinum crispum (parsley).

10. The probiotic of claim 1, wherein nucleic acid encoding the PAL is operably linked to a promoter an expression construct in the probiotic.

11. The probiotic of claim 10, wherein the promoter is a constitutive promoter or an inducible promoter.

12. The probiotic of claims 10 to 11, wherein the promoter is inducible in response to the presence of nisin.

13. The probiotic of any one of claims 10 to 12, wherein the nucleic acid encoding the PAL is codon optimized for expression in the probiotic.

14. A genetically modified probiotic, which is Lactococcus lactis expressing recombinant phenylalanine ammonia lyase (PAL) from Petroselinum crispum (parsley), wherein nucleic acid encoding the PAL is codon optimized for expression in the L. lactis, and wherein the nucleic acid is operably linked to a nisA promoter or functional fragment thereof.

15. The probiotic of claim 1, wherein the PAL is secreted.

16. The probiotic of claim 15, wherein the PAL is expressed as a fusion protein with a secretion signal operable in the probiotic.

17. The probiotic of claim 1, which additionally expresses a chaperone to thereby increase expression of the PAL.

18. The probiotic of claim 1, wherein the probiotic additionally expresses a protein that confers resistance to bile salts.

19. The probiotic of any one of claims 1 to 18, which is encapsulated.

20. A composition comprising the probiotic of any one of claims 1 to 19 and a carrier.

21. A method for reducing levels of phenylalanine in a subject having phenylketonuria (PKU) or preventing an increase in levels of phenylalanine in a subject having PKU after consuming a phenylalanine-containing foodstuff, the method comprising administering to the subject the probiotic of any one of claims 1 to 19 or the composition of claim 20.

22. A method for treating or preventing a symptom of phenylketonuria (PKU) in a subject, the method comprising administering to the subject the probiotic of any one of claims 1 to 19 or the composition of claim 20.
